(19) United States
(12) Patent Application Publication
(10) Pub. No.: US 2026/0078086 A1
(43) Pub. Date: Mar. 19, 2026

(54) VERSATILE BIODEGRADABLE MOLECULES AS NUCLEIC ACID CARRIERS

(71) Applicant: TIBA Biotech LLC, Cambridge, MA (US)

(72) Inventors: Poulami Talukder, Medford, MA (US); Maria Varghese, Wilmington, MA (US); Nathan Ivanowsky, Medford, MA (US)

(21) Appl. No.: 19/327,796

(22) Filed: Sep. 12, 2025

Related U.S. Application Data
(60) Provisional application No. 63/694,619, filed on Sep. 13, 2024.

(51) Int. Cl. C07C 321/14; A61K 9/51; A61K 31/7105; A61K 39/385; C07C 321/18; C07F 5/02

(52) U.S. Cl. CPC C07C 321/14; A61K 9/5123; A61K 9/5146; A61K 31/7105; A61K 39/385; C07C 321/18; C07F 5/027

(57) ABSTRACT

Disclosed are compounds useful for delivering therapeutic or immunogenic nucleic acid agents. Further disclosed are nanoparticle compositions having such a compound and methods for treating or preventing a disease or condition.

### CROSS REFERENCE TO RELATED APPLICATION

This application claims benefit of and priority to U.S. Application No. 63/694,619 filed on Sep. 13, 2024, the entire content of which is incorporated by reference.

### FIELD

The disclosure relates to dendrimer or dendron carriers for efficient delivery of nucleic acids to a subject for treating or preventing diseases or disorders.

### BACKGROUND

Polyester dendrimers as delivery systems have a number of useful properties, such as monodispersity, low reactogenicity, simplicity, structural versatility, good cytotoxicity profile, and degradation into non-toxic adducts and a relatively low cost of synthesis. See Carlmark et al., Chem. Soc. Rev. 42, 5858-79 (2013); Talukder et al., US 20230149562A1; and US 2024/0207187 A1.

Disulfide compounds have also been used in delivery systems, allowing controlled release of therapeutic cargos. See Jiang et al., Isr. J. Chem. 60, 132-139 (2020). However, disulfide compounds are generally not suitable for delivering nucleic acids in an efficient manner because of their poor stability in the presence of reducing agents and potential in vivo side reactions with proteins in the surrounding environment (Talebian et al., 2019). The position of the biodegradable linkage (e.g., ester and disulfide) in the molecule plays an important role in the delivery efficiency, most likely by altering susceptibility to cleavage to low molecular weight compounds. The cleavage of disulfide-based linkages in an appropriate intracellular compartment (e.g., the reducing environment of the cytosol) may contribute to improved payload release in that compartment, if such cleavage is not premature outside that compartment. It is a substantial challenge in the design of nucleic acid carriers that balance the degradability of a linkage with the molecule's overall stability and ability to produce nanoparticles surviving in vivo administration and thus reaching targeted cells.

There is a need to develop compounds that efficiently deliver nucleic acids and also readily degrade to low molecular weight (MW) compounds at the desired site of action.

### SUMMARY

It is unexpectedly found that certain dendrimers and dendrons are highly biodegradable and also very efficient in delivering nucleic acids.

Accordingly, one aspect of this invention relates to compounds of formula I as nucleic acid carriers:

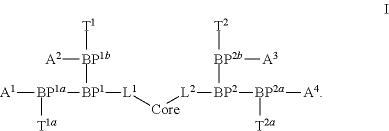

In this formula,
(i) Core is disulfide, heteroaryl, or C₁-C₆ alkylene unsubstituted or substituted with one or more of carboxyl (—COOH), carboxylate (—C(O)—O—), carbamate (

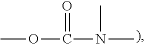

and carbamide

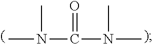

(ii) each of L¹ and L², independently, is C₁-C₃ alkylene;
(iii) each of BP¹ and BP², independently, is a bond, disulfide, O, S, NH, C₁-C₆ alkyl-N, N,

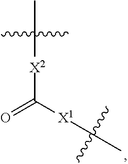

X¹ being a bond, NH, C₁-C₆ alkyl-N, O, S, or SS, X² being NH, C₁-C₆ alkyl-N, N, O, S, SS, SS—(CH₂)₁₋₂₀O,

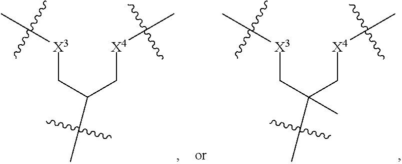

and each of X³ and X⁴, independently, being NH, C₁-C₆ alkyl-N, O, S, SS, or SS—(CH₂)₁₋₂₀O;
(iv) each of BP^{1a} and BP^{2a} is absent, a bond, disulfide, O, S, NH, C₁-C₆ alkyl-N, N, or

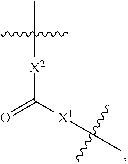

X¹ being a bond, NH, C₁-C₆ alkyl-N, O, S, or SS, X² being NH, C₁-C₆ alkyl-N, N, O, S, SS, SS—(CH₂)₁₋₂₀O,

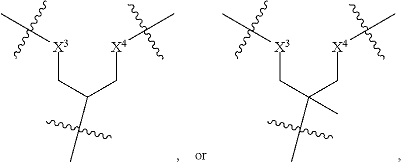

and each of X³ and X⁴, independently, being NH, C₁-C₆ alkyl-N, O, S, SS, or SS—(CH₂)₁₋₂₀O; (v) each of BP^{1b} and BP²¹ is a bond, disulfide, O, S, NH, C₁-C₆ alkyl-N, N, or

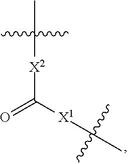

X¹ being a bond, NH, C₁-C₆ alkyl-N, O, S, or SS, X² being NH, C₁-C₆ alkyl-N, N, O, S, SS, SS—(CH₂)₁₋₂₀O,

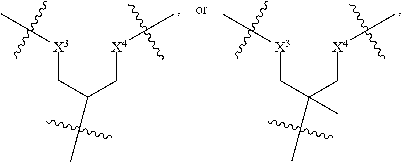

and each of X³ and X⁴, independently, being NH, C₁-C₆ alkyl-N, O, S, SS, or SS—(CH₂)₁₋₂₀O; (vi) each of T^{1a} and T^{2a}, independently, is absent,

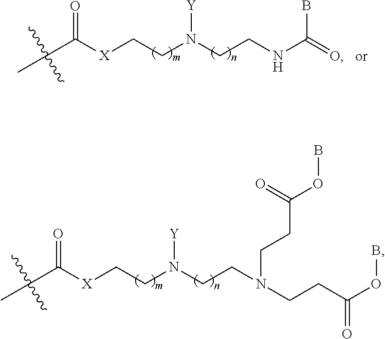

wherein B is a hydrophobic unit; X is NH, O, or S; Y is H, CH₃, C₂H₅, C₃H₇, CH₂CH₂OH, CH₂CH₂CH₂OH, CH₂CH₂NMe₂, CH₂CH₂CH₂NMe₂, CH₂CH₂O(CO)NHMe, or CH₂CH₂CH₂O(CO)NHMe; and each of m and n, independently, is 0 to 20,
(vii) each of T¹ and T², independently, is absent,

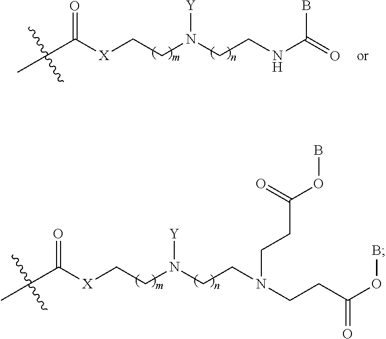

and
(viii) each of A¹, A², A³, and A⁴, independently, is absent, an amine,

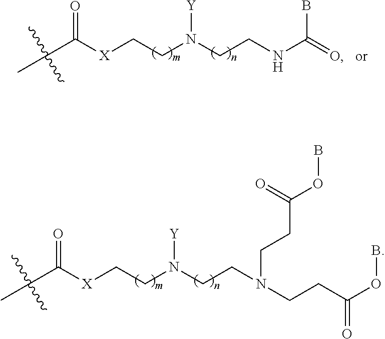

In a preferred embodiment, (i) Core is S—S; (ii) each of L¹ and L², independently, is C₁-C₃ alkylene; (iii) BP¹ is a bond or

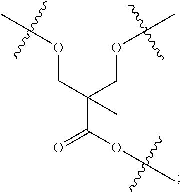 [SMILES: *OCC(C)(CO*)C(=O)O*]

### (iv) BP² is

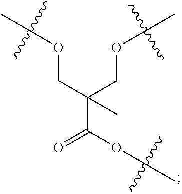 [SMILES: *OCC(C)(CO*)C(=O)O*]

(v) BP^{1b} is absent, a bond, or

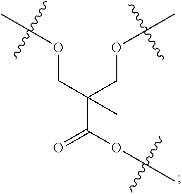 [SMILES: *OCC(C)(CO*)C(=O)O*]

(vi) each of BP^{1a}, BP^{2a} and BP^{2b} is a bond, or

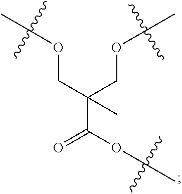 [SMILES: *OCC(C)(CO*)C(=O)O*]

(vii) each of T¹ and T^{1a}, independently, is absent,

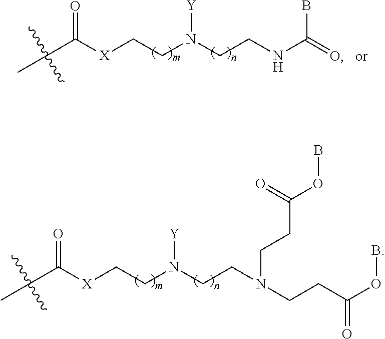

wherein B is a hydrophobic unit; X is NH, O, or S; Y is H, CH₃, C₂H₅C₃H₇, CH₂CH₂OH, CH₂CH₂CH₂OH, CH₂CH₂NMe₂, CH₂CH₂CH₂NMe₂, CH₂CH₂O(CO)NHMe, or CH₂CH₂CH₂O(CO)NHMe; and each of m and n, independently, is 0 to 20, (viii) each of T² and T^{2a}, independently, is

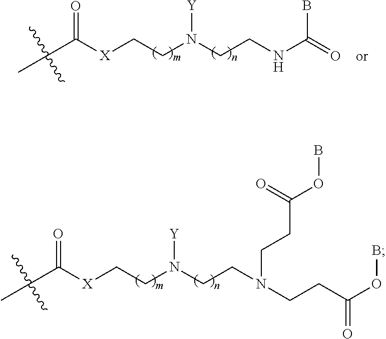

and (ix) each of A¹, A², A³, and A⁴, independently, is absent, an amine,

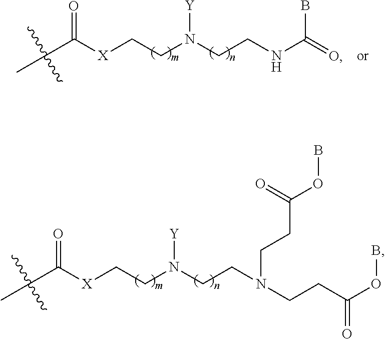

provided that (a) when each of BP^{1a} and BP^{1b} is a bond, A¹ and A² are absent and T^{1a} is

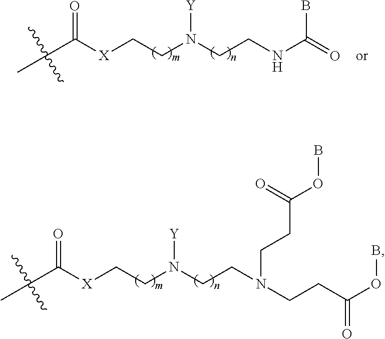

and (b) when each of BP^{2a} and BP^{2b} is a bond, A³ and A⁴ are absent and T^{2a} is

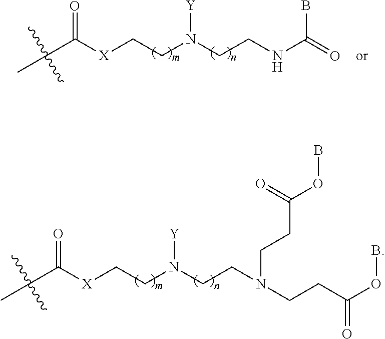

Preferred subsets of the compounds of formula I can have one or any combinations of the following features:
(1) each of L¹ and L², independently, is methylene or ethylene;
(2) Core is disulfide selected from the group consisting of SS,

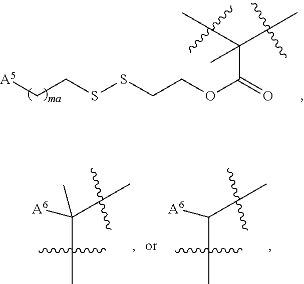

A⁵ and A⁶ being defined below;
(3) Core is SS,

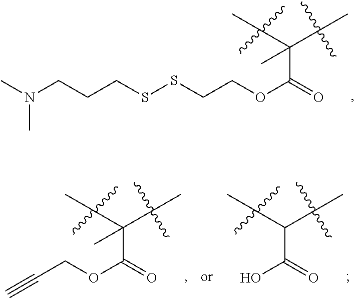

(4) each of BP¹ and BP² is

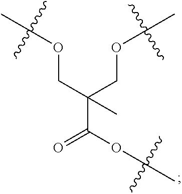 [SMILES: *OCC(C)(CO*)C(=O)O*]

(5) each of BP¹ and BP², independently, is a bond, SS—(CH₂)₁₋₂₀O, or O;
(6) each of BP^{1a} and BP^{2a}, independently, is absent or a bond;
(7) each of BP^{1b} and BP^{2b} is a bond;
(8) each of T¹, T^{1a}, T², and T^{2a}, independently, is

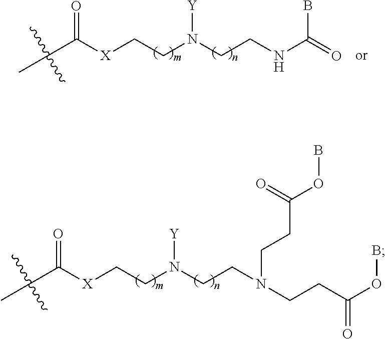

(9) each of A¹, A², A³, and A⁴ is absent;
(10) each of T^{1a} and T^{2a} is absent, and each of T¹, T^{1a}, T², T^{2a}, independently, is

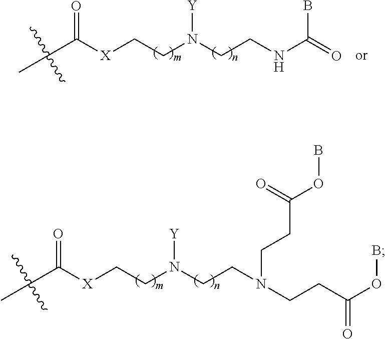

(11) each of T¹, T^{1a}, T², and T^{2a} is

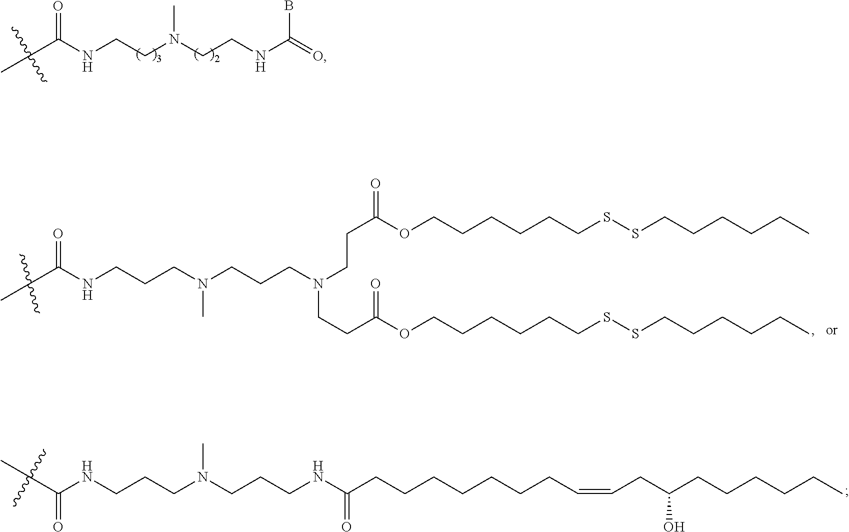

(12) each of T^{1a} and T^{2a} is absent, and each of T¹ and T² is

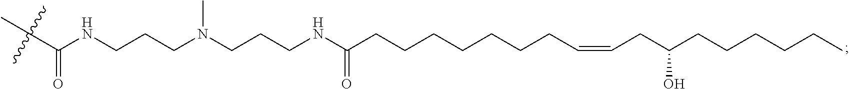 [SMILES: *C(=O)NCCCN(C)CCCNC(=O)CCCCCCC/C=C\C[C@@H](O)CCCCCC]

(13) X is NH;
(14) Y is CH₃;
(15) B is a ricinoleyl moiety;
(16) B is disulfanyl tail with specific formula Z1-S—S—Z2, where Z1 is C₄-C₁₀ alkylene, and Z2 is C₆-C₁₂ alkyl or alkenyl;
(17) B is derived from a fatty acid or derivative thereof that is selected from the group consisting of: caprylic acid, capric acid, lauric acid, myristic acid, palmitic acid, stearic acid, oleic acid, linoleic acid, linolenic acid, arachidonic acid, eicosapentanoic acid, 12-hydroxy-9-cis-octadecenoic acid, 12-methyltetradecanoic acid, 12-methyltridecanoic acid, 14-methylhexadecanoic acid, 14-methylhexadecanoic acid, 18-methylnonadecanoic acid, 19-methylarachidic acid, isopalmitic acid, isostearic acid, phytanic acid, (±)-2-hydroxyoctanoic acid, (±)-3-hydroxydecanoic acid, (±)-3-hydroxyoctanoic acid, 10-hydroxydecanoic acid, 12-hydroxyoctadecanoic acid, 15-hydroxypentadecanoic acid, 16-hydroxyhexadecanoic acid, 2-hydroxyhexadecanoic acid, 2-hydroxytetradecanoic acid, 2-hydroxydodecanoic acid, DL-α-hydroxystearic acid, DL-β-hydroxylauric acid, DL-β-hydroxymyristic acid, and DL-β-hydroxypalmitic acid. Optionally the fatty acid comprises one or more stable isotopes such as a stable isotope of carbon (¹³C) or hydrogen (²H). Examples include octanoic acid-1-¹³C, octanoic acid-8-¹³C, octanoic acid-8,8,8-d3, octanoic-²H15 acid, decanoic acid-1-¹³C, decanoic acid-10-¹³C, decanoic-10,10,10-d3 acid, decanoic-d19 acid, undecanoic acid-1-¹³C, lauric acid-12,12,12-²H3, lauric-²H23 acid, lauric acid-1-¹³C, lauric acid-1,12-¹³C₂, tridecanoic-2,2-²H2 acid, myristic acid-14-¹³C, myristic acid-1-¹³C, myristic acid-14,14,14-²H3, myristic-²H27 acid, palmitic acid-1-¹³C, palmitic acid-16-¹³C, palmitic acid-16-¹³C,16,16,16-²H3, palmitic acid-²H31, stearic acid-1-¹³C, stearic acid-18-¹³C, stearic acid-18,18,18-²H3, stearic-²H35 acid, oleic acid-1-¹³C, oleic acid-²H34, linolenic acid-1-¹³C, linoleic acid-²H32, arachidonic-5,6,8,9,11,12,14,15-²H8 acid, and eicosanoic-²H39 acid;
(18) m is 3 and n is 2.

Further subsets of the compounds of formula I are compounds of formula 1a, 1b, 2a, 2b, 3a, 3b, 4a and 4b:

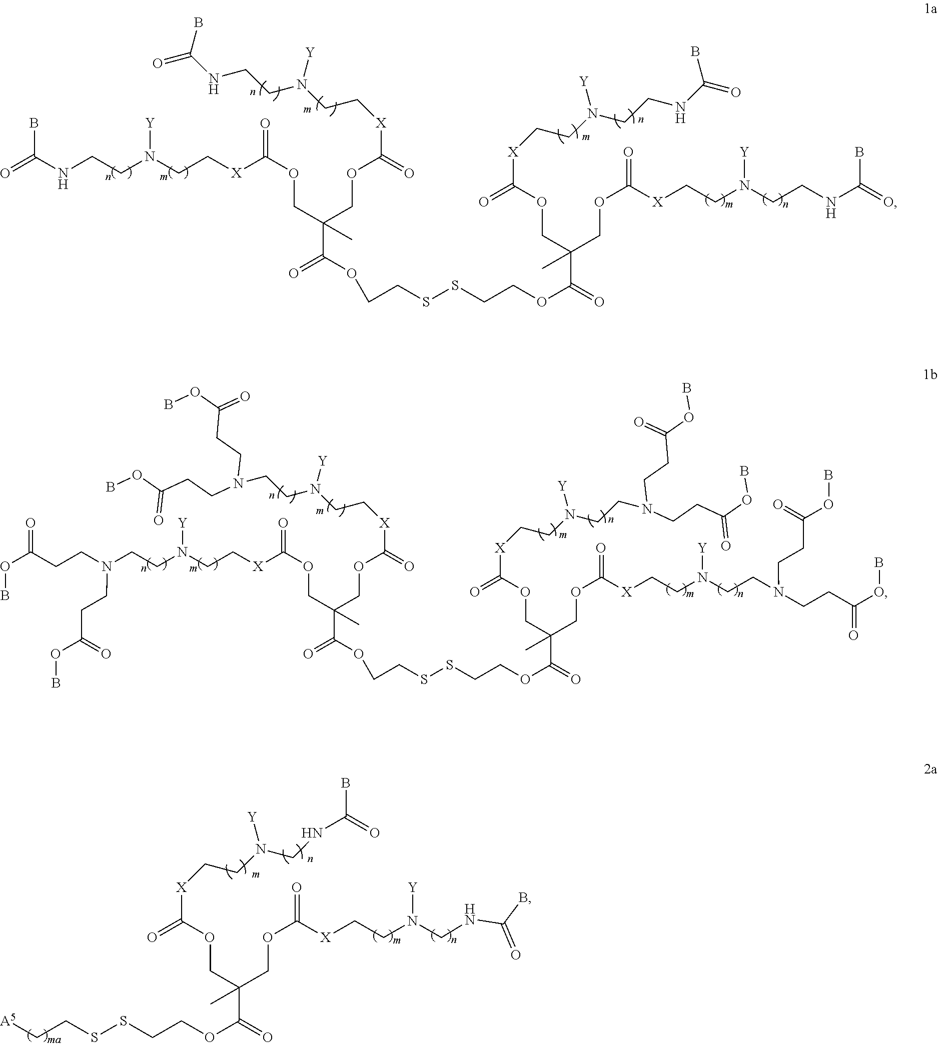

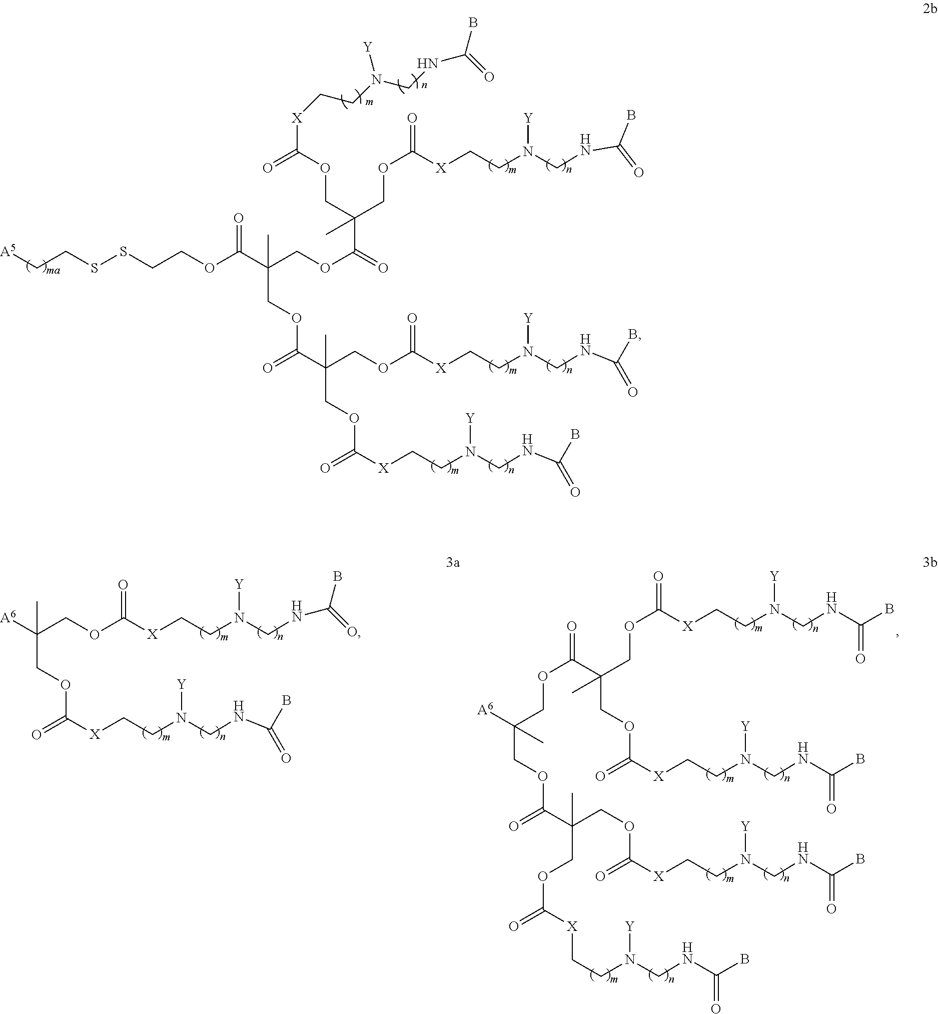

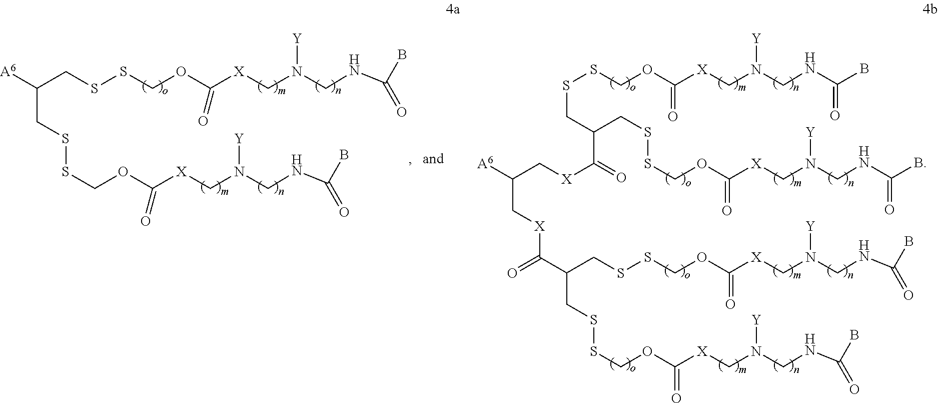

In the formulas above, each of m, n, and O, independently, is an integer from 0 to 20, preferably 1 to 5; A⁶ is T¹, COOH,

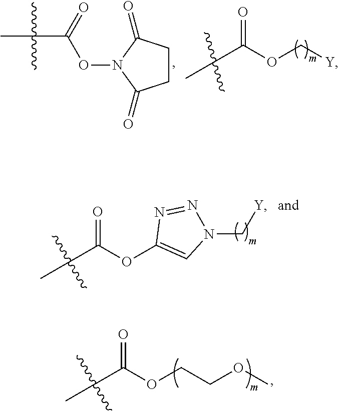

wherein Y is selected from methyl, iso-propyl, sec-butyl, iso-butyl, tert-butyl, isopentyl, neopentyl, 3-methylhexyl, 2,2-dimethylpentyl, 2,3-dimethylpentyl, azide (N3), halogen (Cl, Br, or I), acetylene (C₂H₂), hydroxyl (—OH), or thiol (—SH), -pyranosyl, cycloalkyl, aryl, heteroaryl, and heterocycle; A is an amine linker; B is a hydrophobic unit; and m is 1 to 20; preferably wherein the cycloalkyl, aryl, heteroaryl, and heterocycle are substituted with at least one group selected from halogen, hydroxyl (—OH) and alkyl group.

Exemplary carriers of this invention include:

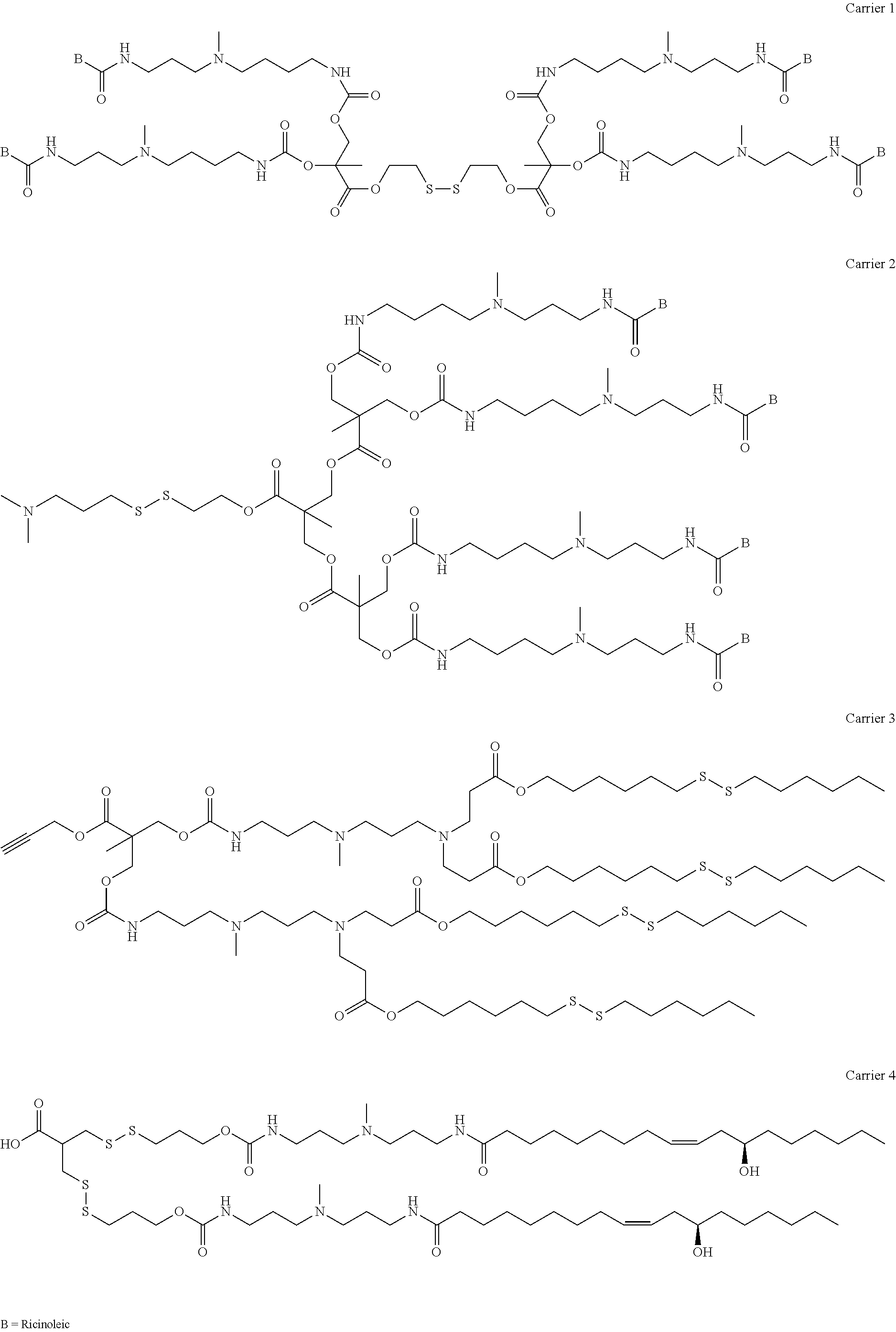

Another aspect of the invention relates to nanoparticle compositions containing any one of the compounds described above and a nucleic acid agent, wherein the nucleic acid agent is therapeutic or immunogenic, fully or partially encapsulated in the nanoparticle.

Suitable therapeutic or immunogenic nucleic acid agents include a polynucleotide, oligonucleotide, DNA, cDNA, RNA, repRNA, siRNA, miRNA, sgRNA, and mRNA, especially an RNA or DNA capable of silencing, inhibiting or modifying the activity of a gene. Typically, the therapeutic or immunogenic nucleic acid agent encodes one or more proteins or antigens associated with a condition selected from the group consisting of infectious disease, pathogen, cancer, autoimmunity disease and allergenic disease.

The nanoparticle composition can further contain a PEG-lipid, such as 1,2-dimyristoyl-sn-glycero-3-phosphoethanol-amine-N-[methoxy (poly-ethylene glycol)-2000], and 1,2-dimyristoyl-rac-glycero-3-methoxypolyethylene glycol-2000, preferably in a range from 1 mol % to 10 mol % of the PEG-lipid per nanoparticle composition.

In addition, the nanoparticle composition can also contain at least one of a phospholipid, a cholesterol, or a cholesterol derivative.

Examples of the phospholipid are 1,2-dioleoyl-sn-glycero-3-phosphoethanol-amine (DOPE) and distearoylphosphatidylcholine (DSPC).

Preferably, the phospholipid constitutes 10 mol % to 15 mol % of the nanoparticle composition calculated as mol % of phospholipid=(mol of phospholipid/[mol of phospholipid+mol of cholesterol or cholesterol derivative+mol of PEG lipid+mol of nucleic acid carrier+mol of nucleic acid])×100.

The cholesterol or cholesterol derivative can be included in a range from 50 mol % to 75 mol % of the cholesterol or cholesterol derivative per nanoparticle composition where mol % of cholesterol or cholesterol derivative=(mol of cholesterol or cholesterol derivative/[mol of cholesterol or cholesterol derivative+mol of phospholipid+mol of PEG lipid+mol of nucleic acid carrier+mol of nucleic acid])×100.

Also within the scope of this invention is a method for treating or preventing a disease or condition in a subject comprising the step of administering a therapeutically effective amount of any nanoparticle composition described above.

The therapeutically effective amount of the nanoparticle composition preferably has the therapeutic or immunogenic nucleic acid agent in a range from 0.01 mg nucleic acid to 10 mg nucleic acid per kg body weight of the subject.

The subject can be a mammal selected from the group consisting of a rodent, a canine, a primate, an equine, a high-value agricultural animal, and a human.

The current invention further includes use of such a compound (e.g., a nanoparticle composition containing one of the compounds described above) for treating or preventing a disease or condition for the manufacture of a medicament.

The details of one or more embodiments of the invention are set forth in the description below. Other features, objects, and advantages of the invention will be apparent from the description, the drawings, and the claims.

### BRIEF DESCRIPTION OF THE DRAWINGS

The following detailed description of preferred embodiments of the present invention will be better understood when read in conjunction with the appended drawings. For the purpose of illustrating the invention, particular embodiments are shown in the drawings. It is understood, however, that the invention is not limited to the precise arrangements and instrumentalities shown. In the drawings:

FIG. 1 illustrates a photograph of the agarose gel showing the binding of the disulfide-based carrier 1 and carrier 2 with luciferase(Luc) mRNA.

FIG. 2 shows luciferase expression in cell culture from luciferase mRNA delivered into mammalian cells by nanoparticles and measured by quantification of intracellular luciferase activity using a luminescence assay.

FIG. 3 illustrates quantification in vivo of Luciferase expression at different organs after administration of nanoparticle formulations containing Luc mRNA and carrier 1 and carrier 2.

### DETAILED DESCRIPTION

Certain terminology is used in the following description for convenience only and is not limiting.

The term “alkyl” as used herein, means a straight or branched chain hydrocarbon. An alkyl group herein may have from 1 to 28 carbon atoms unless otherwise specified. An alkyl group may have 1, 2, 3, 4, 5, 6, 7, 8, 9, 10, 11, 12, 13, 14, 15, 16, 17, 18, 19, 20, 21, 22, 23, 24, 25, 26, 27, or 28 carbon atoms, or a number of carbon atoms in a range from a first of the foregoing values to a second of the foregoing values, where the first and second values selected are any two of the foregoing values and the first value is less than the second. Alkyl chain length may be used to control the hydrophobicity and self-assembly properties of a nucleic acid carrier. Examples include methyl (Me), ethyl (Et), propyl, isopropyl, butyl, etc.

The term “alkylene” refers to a straight or branched hydrocarbon group, containing 1-20 carbon atoms (e.g., C₁₋₆ and C₁₋₃) and two monovalent radical centers or a bivalent radical center derived by the removal of two hydrogen atoms from one or more carbon atoms of a parent alkane. Examples include —CH₂—, —CH₂CH₂—, —CH(CH₃)—, —C(CH₃)₂—, —CH₂CH₂CH₂—, —CH(CH₃)CH₂—.

The term “alkenyl” or “alkenylene” refers to a linear or branched monovalent or divalent hydrocarbon moiety that contains at least one double bond.

The term “alkoxy” refers to O-alkyl.

The term “carbonyl” refers to —C(O)—R″, in which R″ is (e.g., C₂-C₂₀ and C₄-C₁₆) aliphatic, C₁-C₃₀ (e.g., C₂-C₂₀ and C₄-C₁₆) heteroaliphatic, aryl, or heteroaryl.

The term “carboxyl” refers to —COOH.

The term “carboxylate” refers to —O—C(O)—R″ or —C(O)—O—R″, in which R″ is C₁-C₃₀ (e.g., C₁-C₂₀, C₁-C₁₀, and C₁-C₆) aliphatic, C₁-C₃₀ (e.g., C₁-C₂₀, C₁-C₁₀, and C₁-C₆) heteroaliphatic, aryl, or heteroaryl.

The term “carbamate” refers to —O—C(O)—NR′R″ or —NR′—C(O)—O—R″, in which each of R′ and R″ is H, C₁-C₃₀ (e.g., C₁-C₂₀, C₁-C₁₀, and C₁-C₆) aliphatic, C₁-C₃₀ (e.g., C₁-C₂₀, C₁-C₁₀, and C₁-C₆) heteroaliphatic, aryl, or heteroaryl.

The term “carbamide” refers to a compound or moiety containing the functional group of —NR′—C(O)—NR″—, in which each of R′ and R″ is H, C₁-C₃₀ (e.g., C₁-C₂₀, C₁-C₁₀, and C₁-C₆) aliphatic, C₁-C₃₀ (e.g., C₁-C₂₀, C₁-C₁₀, and C₁-C₆) heteroaliphatic, aryl, or heteroaryl.

The term “alkynyl’ or “alkynylene’ refers to a linear or branched monovalent or divalent hydrocarbon moiety that contains at least one triple bond.

The term “cycloalkyl or “cycloalkylene’ refers to a saturated or unsaturated, cyclic, nonaromatic, monovalent or divalent hydrocarbon moiety, Such as cyclohexyl and cyclohexylene. The term “cycloalkenyl or “cycloalkenylene’ refers to a non-aromatic, cyclic hydrocarbon moiety that contains at least one double bond. The term “cycloalkynyl’ or “cycloalkynylene’ refers to a non-aromatic, cyclic hydrocarbon moiety that contains at least one triple bond.

The term “heterocycloalkyl” refers to a nonaromatic 5-8 membered monocyclic, 8-12 membered bicyclic, or 11-14 membered tricyclic ring system having one or more heteroatoms (e.g., O, N, P, and S). Examples include aziridinyl, azetidinyl, pyrrolidinyl, dihydrofuranyl, tetrahydrofuranyl, tetrahydropyranyl, tetrahydrothiophenyl, tetrahydro-2-H-thiopyran-1,1-dioxidyl, piperazinyl, piperidinyl, morpholinyl, imidazolidinyl, azepanyl, dihydrothiadiazolyl, dioxanyl, and quinuclidinyl. Both “cycloalkyl” and “heterocyclyl” also include fused, bridged, and spiro ring systems. They further include substituted groups such as halocycloalkyl and haloheterocyclyl.

The term “aliphatic” herein refers to a saturated or unsaturated, linear or branched, acyclic or cyclic nonaromatic hydrocarbon moiety. Examples include, but are not limited to alkyl, alkylene, alkenyl, alkenylene, alkynyl, alkynylene, cycloalkyl, cycloalkylene, cycloalkenyl, cycloalkenylene, cycloalkynyl, and cycloalkynylene moieties. The term “heteroaliphatic” herein refers to an aliphatic moiety containing at least one heteroatom (e.g., O, S, and N, and P).

The term “aryl” herein refers to a monocyclic, bicyclic or tricyclic aromatic ring system. Examples include phenyl, biphenyl, 1- or 2-naphthyl, 1,2-dihydronaphthyl, 1,2,3,4-tetrahydronaphthyl, indenyl, and indanyl. Aryl can be unsubstituted or substituted with alkyl, alkenyl, alkynyl, cycloalkyl, heterocycloalkyl, aryl, heteroaryl, amino, ether, and ester, and the like. groups. The term “aralkyl” refers to alkyl substituted with aryl, i.e., aryl-alkyl.

The term “heteroaryl” herein refers to an aromatic monocyclic, bicyclic, tricyclic, and tetracyclic ring system having one or more heteroatoms (such as 0, S or N). Examples include pyridinyl, pyrimidinyl, quinolinyl, isoquinolinyl, indolyl, benzimidazolyl, benzoxazolyl, benzothiophenyl, benzofuranyl, pyrazolyl, triazolyl, oxazolyl, thiadiazolyl, tetrazolyl, oxazolyl, isoxazolyl, carbazolyl, furyl, imidazolyl, thienyl, thiazolyl, and benzothiazolyl.

The term “heterocyclyl” includes heterocycloalkyl and heteroaryl.

The term “amine” refers to derivatives of ammonia wherein one or more hydrogen atoms have been replaced by a substituent such as an alkyl group.

The term “halo” refers to F, Cl, Br, or I.

The term “disulfide” refers to a compound or moiety containing —S—S—. Examples include —S—S—, alkylene-S—S-alkyl, alkylene-S—S-aryl, etc. The terms disulfide and disulfanyl are exchangeable.

Alkyl, alkylene, alkenyl, alkenylene, alkynyl, alkynylene, alkoxy, cycloalkyl, cycloalkylene, heterocyclyl, aliphatic, heteroaliphatic, amino, amido, carbonyl, carboxylate, carbamate, aryl, aralkyl, disulfanyl, phosphate, nucleobase, and sugar mentioned herein include both substituted and unsubstituted moieties, unless specified otherwise. Examples of a substituent include deuterium (D), hydroxyl (OH), halo (e.g., F and Cl), amino (NH2), cyano (CN), nitro (NO2), alkyl, alkenyl, alkynyl, alkoxy, cycloalkyl, acylamino, alkylamino, aminoalkyl, haloalkyl (e.g., trifluoromethyl), heterocyclyl, alkoxycarbonyl, amido, carboxy (COOH), alkanesulfonyl, alkylcarbonyl, alkenylcarbonyl, carbamido, carbamyl, carboxyl, thioureido, thiocyanato, sulfonamido, aryl, arylamino, aralkyl, and heteroaryl. All substitutes can be further substituted.

The term “compound”, when referring to a compound of this invention, also includes its salts, solvates, and prodrugs. The pharmaceutically acceptable salts include those listed in Handbook of Pharmaceutical Salts: Properties, Selection and Use, 2nd Revised Edition, P. H. Stahl and C. G. Wermuth (Eds.), Wiley-VCH, New York, (2011). In addition to pharmaceutically acceptable salts, other salts are contemplated in the invention. They may serve as intermediates in the purification of compounds or in the preparation of other pharmaceutically acceptable salts, or are useful for identification, characterization or purification of compounds of the invention. A solvate refers to a complex formed between an active compound and a pharmaceutically acceptable solvent. Examples of a pharmaceutically acceptable solvent include water, ethanol, isopropanol, ethyl acetate, acetic acid, and ethanolamine. A prodrug refers to a compound that, after administration, is metabolized into a pharmaceutically active drug. Examples of a prodrug include esters and other pharmaceutically acceptable derivatives.

The compounds of the present invention may contain one or more non-aromatic double bonds or asymmetric centers. Each of them occurs as a racemate or a racemic mixture, a single R enantiomer, a single S enantiomer, an individual diastereomer, a diastereomeric mixture, a cis-isomer, or a trans-isomer. Compounds of such isomeric forms are within the scope of this invention. They can be present as a mixture or can be isolated using chiral synthesis or chiral separation technologies.

The term “substitute” refers to the ability to change one functional group, or moiety, of a compound for another functional group or moiety, provided that the valency of all atoms on the parent structure is maintained. The substituted group is interchangeably referred herein as “substitution” or “substituent.” When more than one position in any given structure is substituted with more than one substituent selected from a specified group, the substituent may be either the same or different at every position.

Numerical values or ranges preceded by “about” refer to the explicitly recited numbers, and the numbers within the experimental error of the measure contemplated. Embodiments described with the modifier “about” may be altered to remove “about” in order to form further embodiments herein. Likewise, embodiments described without the modifier “about” may be altered to add “about” in order to form further embodiments herein.

A range expressed as being between two numerical values, one as a low endpoint and the other as a high endpoint, includes the values between the numerical values and the low and high endpoints. Embodiments herein include subranges of a range herein, where the subrange includes a low and high endpoint of the subrange selected from any increment within the range selected from each single increment of the smallest significant figure, with the condition that the high endpoint of the subrange is higher than the low endpoint of the subrange.

Further embodiments herein include replacing one or more “including” or “comprising” in an embodiment with “consisting essentially of” or “consisting of” “Including” and “comprising,” as used herein, are open ended, include the elements recited, and do not exclude the addition of one or more other element. “Consisting essentially of” means that addition of one or more element compared to what is recited is within the scope, but the addition does not materially affect the basic and novel characteristics of the combination of explicitly recited elements. “Consisting of” refers to the recited elements, but excludes any element, step, or ingredient not specified.

The words “a” and “one,” as used in the claims and in the corresponding portions of the specification, are defined as including one or more of the referenced items unless specifically stated otherwise. This terminology includes the words above specifically mentioned, derivatives thereof, and words of similar import. The phrase “at least one” followed by a list of two or more items, such as “A, B, or C” or “A, B, and C” means any individual one of A, B or C as well as any combination thereof.

An embodiment comprises a nanoparticle composition comprising a nucleic acid carrier having the structure of one of formula I, 1a, 1b, 2a, 2b, 3a, 3b, 4a and 4b shown above, or 1c shown below.

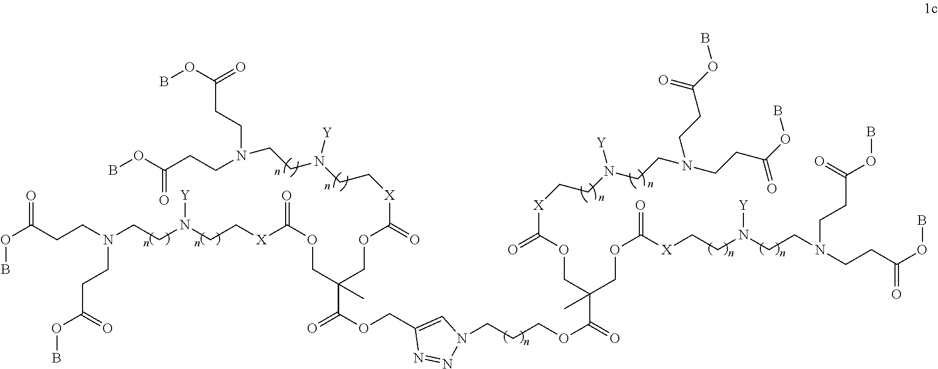

A 2,2-Bis(hydroxymethyl)propionic acid (bis-MPA) based polyester dendrimer or dendron of this invention includes a disulfide core or focal point and a plurality of monomeric polyester units that form one or more generations.

Where B is a hydrophobic unit, X=-NH, —O, —S; Y=-H, —CH₃, —C₂H₅, —C₃H₇, —CH₂CH₂OH, —CH₂CH₂CH₂OH, —CH₂CH₂NMe₂, —CH₂CH₂CH₂NMe₂, —CH₂CH₂—O(CO)NHMe, —CH₂CH₂CH₂O(CO)NHMe; and each of m and n, independently, is 0 to 20.

The amine is a moiety that imparts proton-accepting functionality to the nucleic acid carrier molecule by containing one or more nitrogen atoms with lone pairs. The amine is thus able to accept a free proton (H+) under acidic conditions. In preferred embodiments the nitrogen atom(s) are present in the form of secondary or tertiary amines. The amine may be selected from the following moieties: 1-(2-Aminoethyl)piperazine, 1-Piperidineethanamine, 1-Dimethylamino-2-propylamine, 1-(3-Aminopropyl)pyrrolidine, 1-(2-Aminoethyl)pyrrolidine, 1-(2-Aminoethyl)piperidine, 2-(1-Piperazinyl)ethylamine, 2-(4-Methyl-piperazin-1-yl)-ethylamine, 3-(Dimethylamino)-1-propylamine, 3-(Diethylamino)-propylamine, (4-Aminobutyl)dimethylamine, 4-(1-Pyrrolidinyl)-1-butylamine, 4-Morpholine-ethanamine, 4-Morpholinepropanamine, 4-Methyl-1-piperazineethanamine, 4-(Diethyl-amino)butylamine, N,N-Dimethylethylenediamine, N,N-Dimethyldipropylenetriamine, N,N′-Dimethyl-N,N′-bis(3-methylaminopropyl)trimethylenediamine, and dimethylamine.

The hydrophobic unit B of Formula 1a, 1b, 1c, 2a, 2b, 3a, 3b, 4a and 4b may be a C₁-C₂₈ alkyl or C₂-C₂₈ alkenyl group. Each of the C₁-C₂₈ alkyl or C₂-C₂₈ alkenyl group may be optionally substituted with one to four substituents selected from halogen, CN, NO₂, N₃, C₁-C₆ alkyl, halo(C₁-C₆ alkyl), OR, NR₂, CO₂R, OC(O)R, CON(R)₂, OC(O)N(R)₂, NHC(O)N(R)₂, NHC(NH)N(R)₂, C₃-C₈ cycloalkyl, C₃-C₈ cycloalkenyl, aryl, heteroaryl, or heterocycle. Each R may independently be selected from hydrogen, C₁-C₆ alkyl, halo(C₁-C₆ alkyl), C₃-C₈ cycloalkyl, C₃-C₈ cycloalkenyl, aryl, heteroaryl, or heterocycle. Each cycloalkyl, cycloalkenyl, aryl, heteroaryl, and heterocycle may be further optionally substituted with R′, wherein R′ may independently be selected from halogen, CN, NO₂, N₃, C₁-C₆ alkyl, or halo(C₁-C₆ alkyl). Embodiments herein include a nucleic acid carrier having one or more of the amine linkers.

The hydrophobic unit B of Formula 1a, 1b, 1c, 2a, 2b, 4a and 4b may be introduced by contacting the nucleic acid carrier with a functional reagent such as a fatty acid. The fatty acid may be a saturated or unsaturated fatty acid having C4-C28 chains. The fatty acid may be, but is not limited to, caprylic acid, capric acid, lauric acid, myristic acid, palmitic acid, stearic acid, oleic acid, linoleic acid, linolenic acid, arachidonic acid, eicosapentanoic acid, 12-hydroxy-9-cis-octadecenoic acid (ricinoleic acid), 12-methyltetradecanoic acid, 12-methyltridecanoic acid, 14-methylhexadecanoic acid, 14-methylhexadecanoic acid, 18-methylnonadecanoic acid, 19-methylarachidic acid, isopalmitic acid, isostearic acid, phytanic acid, (±)-2-hydroxyoctanoic acid, (±)-3-hydroxydecanoic acid, (±)-3-hydroxyoctanoic acid, 10-hydroxydecanoic acid, 12-hydroxyoctadecanoic acid, 15-hydroxypentadecanoic acid, 16-hydroxyhexadecanoic acid, 2-hydroxyhexadecanoic acid, 2-hydroxytetradecanoic acid, 2-hydroxydodecanoic acid, DL-α-hydroxystearic acid, DL-β-hydroxylauric acid, DL-β-hydroxymyristic acid, or DL-β-hydroxypalmitic acid. The fatty acid may be selected from conjugated fatty acids (e.g., conjugated isomers of linoleic acid); acetylenic fatty acids (e.g., crepenynic acid); allenic fatty acids (e.g., laballenic acid) or cyclopropenyl fatty acids (e.g., sterculic acid).

The hydrophobic unit B of Formula I, 1a, 1b, 1c, 2a, 2b, 3a, 3b, 4a and 4b may be introduced by contacting the nucleic acid carrier with a functional reagent such as an acrylate. The acrylates may be saturated or unsaturated having C₄-C₂₈ chains. The acrylates may have symmetrical or unsymmetrical disulfides with varying alkyl chain length (C₄-C₂₈).

An embodiment comprises a nanoparticle composition comprising any one or more of the nucleic acid carriers described herein. The nanoparticle composition may further comprise an agent; for example, a nucleic acid. A nanoparticle composition herein may be useful to introduce an agent into a cell. A nanoparticle composition herein may be useful as a transfection agent. A nanoparticle composition herein may be useful in a method of treating or preventing a disease.

In an embodiment, a nanoparticle composition may comprise a mixture of nucleic acid carriers, each one of them comprising different amine and/or side chains. These nucleic acid carriers may be mixed at a fixed ratio. For an example of mixture with three nucleic acid carriers, a ratio of the first nucleic acid carrier to the second nucleic acid carrier and to the third nucleic acid carrier may be i:j:k where i, j, and k are independently selected from 1, 2, 3, 4, 5, 6, 7, 8, 9, 10, 11, 12, 13, 14, 15, 16, 17, 18, 19, or 20, or a value in a range from a first of the foregoing values to a second of the foregoing values, where the first and second values selected are any two of the foregoing values and the first value is less than the second.

In an embodiment, the nanoparticle composition may comprise one or more nucleic acid agents. The nucleic acid agents may be therapeutic or immunogenic. The therapeutic or immunogenic nucleic acid agent may be non-covalently bound or covalently bound to the nucleic acid carrier. The nucleic acid agent may be bound to the charged nucleic acid carrier through electrostatic interaction and hydrogen bonding.

As used herein, the term “nucleic acid” refers to any natural or synthetic DNA or RNA molecules, RNA/DNA hybrids and polyamide nucleic acids (PNAs) all of which can be in single- or double-stranded form, and unless otherwise mentioned, may include known analogs of natural nucleotides that can function in a similar manner as naturally occurring nucleotides. The nucleic acid agent may also be a mixture of one or more different RNA molecules, DNA molecules, or combination of the two. The term “DNA” or “DNA molecule” or “deoxyribonucleic acid molecule” refers to a polymer of deoxyribonucleotides. The DNA molecule may be a polynucleotide, oligonucleotide, DNA, or cDNA. The DNA molecule may encode wild-type or engineered proteins, peptides or polypeptides. The encoded protein, peptide, or polypeptide may be an antigen. The term “RNA” or “RNA molecule” or “ribonucleic acid molecule” refers to a polymer of ribonucleotides. The polymer may have 2, 3, 4, 5, 10, 15, 20, 25, 30, 35, 40, 45, 50, 55, 60, 65, 70, 75, 80, 85, 90, 95, 100, 200, 300, 400, 500, 600,700, 800, 900, 1000, 2000, 3000, 4000, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, or 50000 ribonucleotides, or a number of ribonucleotides in a range from a first of the foregoing values to a second of the foregoing values, where the first and second values selected are any two of the foregoing values and the first value is less than the second. The RNA molecule may be a replicon RNA (repRNA), small interfering RNA (siRNA), miRNA, single strand guide RNA (sgRNA), messenger RNA (mRNA), or transfer RNA (tRNA). Replicon RNA (repRNA) refers to a genome replication-competent, progeny-defective RNA virus genome that is incapable of producing infectious progeny virions. Viral genomes that are typically modified for use as repRNAs include “positive strand” RNA viruses. The modified viral genomes function as both mRNA and templates for replication. Small interfering RNA (siRNA) refers to an RNA (or RNA analog) comprising between about 10-50 nucleotides (or nucleotide analogs) which is capable of directing or mediating RNA interference. MicroRNAs (miRNAs) refers to small (20-24 nt) regulatory non-coding RNAs that are involved in post-transcriptional regulation of gene expression in eukaryotes by affecting either or both the stability and translation of coding mRNAs. Messenger RNAs (mRNAs) are usually single-stranded RNAs and define the amino acid sequence of one or more polypeptide chains. This information is translated during protein synthesis when ribosomes bind to the mRNA. The DNA or RNA molecules may be chemically modified in nucleic acid backbone, the ribose sugar moiety and the nucleobase itself.

The RNA molecule may be a monocistronic or polycistronic mRNA. A monocistronic mRNA refers to an mRNA comprising only one sequence encoding a protein, polypeptide or peptide. A polycistronic mRNA typically refers to two or more sequences encoding two or more proteins, polypeptides or peptides. An mRNA may encode a protein, polypeptide, or peptide.

As used herein, “encapsulated” can refer to a nanoparticle that provides an active agent or therapeutic agent, such as a nucleic acid (e.g., a messenger RNA), with full encapsulation, partial encapsulation, or both. In a preferred embodiment, the nucleic acid is fully encapsulated in the nanoparticle. In the context of nucleic acid therapeutic agents, full encapsulation may be determined by a RiboGreen® assay. RiboGreen® is an ultra-sensitive fluorescent nucleic acid stain for quantitating oligonucleotides and single-stranded DNA or RNA in solution (available from Thermo Fisher Scientific—US).

The use of a “nucleic acid carrier” herein as a carrier of nucleic acids is preferred and the name “nucleic acid carrier” is applied for that reason. However, a non-nucleic acid agent may be in an embodiment herein.

In an embodiment, the nanoparticle composition described herein may comprise a lipid conjugate. In an embodiment, the lipid conjugate may be useful in that it may prevent the aggregation of particles. Lipid conjugates that may be in a composition herein include, but are not limited to, polyethylene glycol (PEG)-lipid conjugates. Non-limiting examples of PEG-lipids include, PEG coupled to lipids such as DMG-PEG 2000, PEG coupled to phospholipids such as phosphatidylethanolamine (PEG-PE), PEG conjugated to cholesterol or a derivative thereof, and mixtures thereof. In certain instances, the PEG may be optionally substituted by an alkyl, alkoxy, acyl, or aryl group.

PEGs are classified by their molecular weights; for example, PEG 2000 has an average molecular weight of about 2,000 daltons, and PEG 5000 has an average molecular weight of about 5,000 daltons. PEGs are commercially available from Avanti Polar Lipids. The PEG moiety of the PEG-lipid conjugates described herein may comprise an average molecular weight ranging from 550 daltons to 10,000 daltons.

Phosphatidylethanolamines having a variety of acyl chain groups of varying chain lengths and degrees of saturation can be conjugated to PEG to form the lipid conjugate. Phosphatidylethanolamines are commercially available or can be isolated or synthesized using conventional techniques. The phosphatidylethanolamines may comprise saturated or unsaturated fatty acids with carbon chain lengths in the range of C₁₀ to C₂₀. The phosphatidylethanolamines may comprise mono- or polyunsaturated fatty acids and mixtures of saturated and unsaturated fatty acids. The phosphatidylethanolamines contemplated include, but are not limited to, dimyristoylphosphatidylethanolamine (DMPE), dipalmitoyl-phosphatidylethanol amine (DPPE), dioleoylphosphatidylethanolamine (DOPE), and distearoyl-phosphatidylethanolamine (DSPE).

The PEG-lipid may comprise PEG conjugated to cholesterol or cholesterol derivative. Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof.

The nanoparticle composition may contain 10 mol % or less of the PEG-lipid per nanoparticle composition. The nanoparticle composition may comprise about 10 mol %, about 9 mol %, about 8 mol %, about 7 mol %, about 6 mol %, about 5 mol %, about 4 mol %, about 3 mol %, about 2 mol %, or about 1 mol %, or an amount in a range from a first of the foregoing integers to a second of the foregoing integers of the PEG-lipid per nanoparticle composition.

The nanoparticle composition may contain “amphipathic lipid”. As used herein, “amphipathic lipid” refers to any material having non-polar hydrophobic units or “tails”, and polar “heads.” Polar groups may include, but are not limited to, phosphate, carboxylic, sulfate, amino, sulfhydryl, nitro, and hydroxyl. Nonpolar groups may include, but are not limited to, long-chain saturated and unsaturated aliphatic hydrocarbon groups and such groups substituted by one or more cycloalkyl, cycloalkenyl, aryl, heteroaryl, or heterocycle group(s). Examples of amphipathic lipids include, but are not limited to, phospholipids, aminolipids, and sphingolipids. Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine. Representative examples of the phosphatidylcholine include, but are not limited to, dipalmitoylphosphatidyl choline, dioleoylphosphatidylcholine, distearoylphosphatidylcholine, and dilinoleoylphosphatidylcholine. Representative examples of the phosphatidylethanolamine include, but are not limited to, 1,2-dioleoyl-sn-glycero-3-phosphoethanolamine or DOPE.

The nanoparticle composition may contain the amphipathic lipid in the amount ranging from 10 mol % to 15 mol % per nanoparticle composition. The amphipathic mol % may be 10, 11, 12, 13, 14, or 15 mol % or a value in a range from a first of the foregoing values to a second of the foregoing values, where the first and second values selected are any two of the foregoing values and the first value is less than the second.

In an embodiment, the nanoparticle composition may include cholesterol or a cholesterol derivative. Examples of cholesterol derivatives include, but are not limited to, cholestanol, 5,6-epoxy cholestanol, cholestanone, cholestenone, coprostanol, cholesteryl-2′-hydroxyethyl ether, cholesteryl-4′-hydroxybutyl ether, 24-ethyl cholesterol, 24-methyl cholesterol, cholenic Acid, 3-hydroxy-5-cholestenoic Acid, cholesteryl palmitate, cholesteryl arachidonate, cholesteryl arachidate, cholesteryl myristate, cholesteryl palmitoleate, cholesteryl lignocerate, cholesteryl oleate, cholesteryl stearate, cholesteryl erucate, cholesterol α-linolenate, cholesteryl linoleate, cholesteryl homo-7-linolenate, 4-hydroxy cholesterol, 6-hydroxy cholesterol, 7-hydroxy cholesterol, 19-hydroxy cholesterol, 20-hydroxy cholesterol, 22-hydroxy cholesterol, 24-hydroxy cholesterol, 25-hydroxy cholesterol, 27-hydroxy cholesterol, 27-alkyne cholesterol, 7-keto cholesterol, 7-dehydro cholesterol, 8-dehydro cholesterol, 24-dehydro cholesterol, 5α-hydroxy-6-keto cholesterol, 20,22-dihydroxy cholesterol, 7,25-dihydroxy cholesterol, 7,27-dihydroxy cholesterol, 7-keto-25-hydroxy cholesterol, fucosterol, phytosterol, cholesteryl 11,14-eicosadienoate, dimethyl hydroxyethyl aminopropane carbamoyl cholesterol iodide and mixtures thereof. The cholesterol derivative may comprise a sugar moiety and/or one or more amino acids. Exemplary, non-limiting sugars for a cholesterol derivative include glucose, mannose, galactose, fructose, sucrose, lactose, trehalose. Exemplary, non-limiting, amino acids for a cholesterol derivative include serine, threonine, lysine, histidine, arginine. The nanoparticle composition may include the cholesterol or cholesterol derivative in an amount ranging from 50 mol % to 75 mol % per nanoparticle composition. The cholesterol or cholesterol derivative mol % may be 50, 51, 52, 53, 54, 55, 56, 57, 58, 59, 60, 61, 62, 63, 64, 65, 66, 67, 68, 69, 70, 71, 72, 73, 74, or 75 mol % or a value in a range from a first of the foregoing values to a second of the foregoing values, where the first and second values selected are any two of the foregoing values and the first value is less than the second.

In an embodiment, a nanoparticle composition; e.g., a pharmaceutical composition herein, may be sterilized by conventional, well-known sterilization techniques. Aqueous solutions may be packaged for use or lyophilized. The lyophilized preparation may be combined with a sterile aqueous solution prior to administration. In an embodiment, a nanoparticle composition may include a pharmaceutically acceptable carrier. As used herein, the term “pharmaceutically-acceptable carrier” means a pharmaceutically acceptable material, composition or vehicle, for example a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier is “pharmaceutically-acceptable” in the sense of being compatible with the other ingredients of the formulation and not injurious to the subject. Some examples of materials which may serve as pharmaceutically-acceptable carriers include: (1) sugars, for example lactose, glucose, mannose and/or sucrose; (2) starches, for example corn starch and/or potato starch; (3) cellulose, and its derivatives, for example sodium carboxymethyl cellulose, methylcellulose, ethyl cellulose, microcrystalline cellulose and/or cellulose acetate; (4) powdered tragacanth; (5) malt; (6) gelatin; (7) lubricating agents, for example magnesium stearate, sodium lauryl sulfate and/or talc; (S) excipients, for example cocoa butter and/or suppository waxes; (9) oils, for example peanut oil, cottonseed oil, safflower oil, sesame oil, olive oil, corn oil and/or soybean oil; (10) glycols, for example propylene glycol; (11) polyols, for example glycerin, sorbitol, and/or mannitol; (12) esters, for example glycerides, ethyl oleate and/or ethyl laurate; (13) agar; (14) buffering agents, for example magnesium hydroxide and/or aluminum hydroxide; (15) alginic acid; (16) pyrogen-free water; (17) diluents, for example isotonic saline, and/or PEG400; (I8) Ringer's solution; (19) C2-C12 alcohols, for example ethanol; (20) fatty acids; (21) pH buffered solutions; (22) bulking agents, for example polypeptides and/or amino acids (23) serum component, for example serum albumin, HDL and LDL; (24) surfactants, for example polysorbates (Tween 80) and/or poloxamers; and/or (25) other non-toxic compatible substances employed in pharmaceutical formulations: for example, fillers, binders, wetting agents, coloring agents, release agents, coating agents, sweetening agents, flavoring agents, perfuming agents, preservatives and/or antioxidants. The terms “excipient,” “pharmaceutically acceptable carrier.” or the like are used interchangeably herein.

An embodiment comprises a method for treating or preventing a disease or condition in a subject. The method may comprise providing any one of the nanoparticle compositions or pharmaceutical compositions described herein. The method may comprise administering a therapeutically effective amount of the nanoparticle composition to a subject.

An embodiment comprises a method for delivering a nucleic acid to a subject. The method may comprise administering any one or more of the nanoparticle compositions herein to a subject. The method may comprise administering a delivery effective amount of any one or more of the nanoparticle compositions herein to a subject. As used herein, a “delivery effective amount” is an amount sufficient to result in a detectable level of expressed product. The delivery effective amount may be a therapeutically effective amount.

As used herein, the term “therapeutically effective amount” refers to the amount of nanoparticle composition which is effective for producing a desired therapeutic effect. The therapeutic effect may be achieved at a reasonable benefit/risk ratio applicable to medical treatment. A “therapeutically effective amount” may refer to an amount sufficient to generate appearance of antigen-specific antibodies in serum. A “therapeutically effective amount” may refer to an amount sufficient to cause a decrease in disease symptoms. A “therapeutically effective amount” may refer to an amount sufficient to cause a disappearance of disease symptoms. When treating viral infection, a decrease of disease symptoms may be assessed by decrease of virus in feces, in bodily fluids, or in secreted products. The nanoparticle compositions may be administered using an amount and by a route of administration effective for generating an immune response.

Therapeutic efficacy may depend on effective amounts of active agents and time of administration necessary to achieve a desired result. Administering a nanoparticle composition may be a preventive measure. Administering of a nanoparticle composition may be a therapeutic measure to promote immunity to the infectious agent, to minimize complications associated with the slow development of immunity especially in patients with a weak immune system, the elderly, or infants.

The exact dosage may be chosen by the clinician based on a variety of factors and in view of individual patients. Dosage and administration may be adjusted to provide sufficient levels of the active agent or agents or to maintain the desired effect. For example, factors which may be taken into account may include the type and severity of a disease; age and gender of the patient; drug combinations; and an individual response to therapy.

Therapeutic efficacy and toxicity of active pharmaceutical agents in a nanoparticle composition may be determined by standard pharmaceutical procedures, for example, by determining the therapeutically effective dose in 50% of the population (ED50) and the lethal dose to 50% of the population (LD50) in cells cultured in vitro or experimental animals. Nanoparticle compositions may be evaluated based on the dose ratio of toxic to therapeutic effects (LD50/ED50), called the therapeutic index, the large value of which may be used for assessment. The data obtained from cell and animal studies may be used in formulating a dosage for human use.

The therapeutically effective dose may be estimated initially from cell culture assays. A therapeutically effective dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage may be monitored by a suitable bioassay.

The amount of particles administered will depend upon the particular therapeutic agent (e.g., nucleic acid) used, the disease or disorder being treated, the age, weight, and condition of the patient, and the judgment of the clinician. A therapeutically effective amount may be a dose from 0.001 ng to 50 mg of the therapeutic or immunogenic nucleic acid per kilogram of body weight of the subject. The therapeutic and immunogenic nucleic acid may be a combination of different nucleic acids used per treatment dose.

The terms “subject” means a human or animal. Preferably, the animal is a vertebrate such as a primate, rodent, domestic animal or game animal. Primates include chimpanzees, cynomolgus monkeys, spider monkeys, and macaques, e.g., Rhesus. The rodent may be selected from mice, rats, guinea pigs, woodchucks, ferrets, rabbits and hamsters. The domestic or game animals may be selected from cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. A patient or subject may be selected from the foregoing or a subset of the foregoing. A patient or subject may be selected from all of the above, but excluding one or more groups or species such as humans, primates or rodents. In an embodiment, the patient or subject may be a mammal, e.g., a primate, e.g., a human. The terms, “patient” and “subject” are used interchangeably herein. Preferably, the subject is a mammal. The mammal may be a human, non-human primate, mouse, rat, dog, cat, horse, cow, or swine but is not limited to these examples. Mammals other than humans may be subjects that represent animal models of a disease or disorder. In addition, the methods described herein may be directed to treating domesticated animals and/or pets. A subject may be male or female.

As used herein, the terms “administer,” “administering,” “administration,” or the like refer to the placement of a composition into a subject. The administration may be by a method or route which results in at least partial localization of the composition at a desired site. Placement at a desired site may lead to a production of a desired effect. A nanoparticle composition described herein may be administered by any appropriate route known in the art including, but not limited to, oral or parenteral routes, including intravenous, intramuscular, subcutaneous, transdermal, airway (aerosol), pulmonary, nasal, rectal, topical, buccal, or sublingual administration.

Exemplary modes of administration include, but are not limited to, injection, infusion, instillation, inhalation, or ingestion. “Injection” includes without limitation, intravenous, intramuscular, intraarterial, intrathecal, intraventricular, intracapsular, intraorbital, intracardiac, intradermal, intraperitoneal, trans tracheal, subcutaneous, subcuticular, intraarticular, sub capsular, subarachnoid, intraspinal, intracerebral, and intrasternal injection and infusion. In an embodiment, the compositions may be administered by intravenous infusion or injection.

The nanoparticle compositions may be used for delivery of therapeutic or immunogenic nucleic acids for gene targeting or modulating gene expression. The therapeutic or immunogenic nucleic acid may be an antisense oligonucleotide (AON) or a double-stranded small interfering RNA (siRNA). Typically, siRNAs are between 21 and 23 nucleotides in length. The siRNAs may comprise a sequence complementary to a sequence contained in an mRNA transcript of a target gene when expressed within the host cell. The antisense oligonucleotide may be a morpholino antisense oligonucleotide. The antisense oligonucleotide may include a sequence complementary to a sequence contained in an mRNA transcript of a target gene. The therapeutic or immunogenic nucleic acid may be an interfering RNA (iRNA) against a specific target gene within a specific target organism. The iRNA may induce sequence-specific silencing of the expression or translation of the target polynucleotide, thereby down-regulating or preventing gene expression. The iRNA may completely inhibit expression of the target gene. The iRNA may reduce the level of expression of the target gene compared to that of an untreated control. The therapeutic or immunogenic nucleic acid may be a microRNA (miRNA). The miRNA may be a short RNA, e.g., a hairpin RNA (hpRNA). The miRNA may be cleaved into biologically active dsRNA within the target cell by the activity of the endogenous cellular enzymes. The RNA may be a double stranded RNA (dsRNA). The ds RNA may be at least 25 nucleotides in length or may be longer. The dsRNA may contain a sequence that is complementary to the sequence of the target gene or genes. An embodiment comprises use of a nanoparticle composition for gene targeting in a subject. An embodiment comprises a method of gene targeting comprising administering a nanoparticle composition herein to a subject.

In an embodiment, the therapeutic or immunogenic nucleic acid may be or may encode an agent that totally or partially reduces, inhibits, interferes with, or modulates the activity or synthesis of, one or more genes encoding target proteins. The target genes may be any genes included in the genome of a host organism. The sequence of the therapeutic or immunogenic nucleic acid may not be 100% complementary to the nucleic acid sequence of the target gene.

In an embodiment, the nanoparticle composition may be used for targeted, specific alteration of the genetic information in a subject. An embodiment comprises targeted, specific alteration of the genetic information in a subject comprising administration of a nanoparticle composition herein. As used herein, the term “alteration” refers to any change in the genome in the cells of a subject. The alteration may be insertion or deletion of nucleotides in the sequence of a target gene. “Insertion” refers to addition of one or more nucleotides to a sequence of a target gene. The term “deletion” refers to a loss or removal of one or more nucleotides in the sequence of a target gene. The alteration may be correction of the sequence of a target gene. “Correction” refers to alteration of one or more nucleotides in the sequence of a target gene, e.g., by insertion, deletion or substitution, which may result in a more favorable expression of the gene manifested by improvements in genotype and/or phenotype of the host organism. An embodiment comprises use of a nanoparticle composition herein for targeted, specific alteration of the genetic information in a subject. An embodiment comprises a method of targeted, specific alteration of the genetic information in a subject comprising administering a nanoparticle composition herein to the subject. An embodiment comprises use of a nanoparticle composition herein for the alteration of the genetic information in the cells of a subject ex vivo by administration of the nanoparticle composition directly to the solution in which the subject's cells are cultured or suspended.

The alteration of the genetic information may be achieved via the genome editing techniques. As used herein, “genome editing” refers to the process of modifying the nucleotide sequence in the genome in a precise or controlled manner.

An exemplary genome editing system is a Clustered Regularly Interspaced Short Palindromic Repeats (CRISPR) system as described, for example, in WO 2018/154387, which published Aug. 30, 2018 and is incorporated herein by reference as if fully set forth. In general, “CRISPR system” refers to transcripts and other elements involved in the expression of CRISPR-associated (Cas) genes, including sequences encoding a Cas gene, a tracr (trans-activating CRISPR) sequence, a tracr-mate sequence, a guide sequence, or other sequences and transcripts from a CRISPR locus. One or more tracr mate sequences may be operably linked to a guide sequence before processing or crRNA after processing by a nuclease. The tracrRNA and crRNA may be linked and may form a chimeric crRNA-tracrRNA hybrid where a mature crRNA is fused to a partial tracrRNA via a synthetic stem loop to mimic the natural crRNA:tracrRNA duplex as described in Cong et al., Science, 15:339(6121):819-823 (2013) and Jinek et al., Science, 337(6096):816-21 (2012), which are incorporated herein by reference as if fully set forth. A single fused crRNA-tracrRNA construct is also referred herein as a guide RNA or gRNA, or single-guide RNA (sgRNA). Within an sgRNA, the crRNA portion is identified as the “target sequence” and the tracrRNA is often referred to as the “scaffold.” In an embodiment, the nanoparticle compositions described herein may be used to deliver an sgRNA.

In an embodiment, the nanoparticle compositions may be used to apply other exemplary genome editing systems including meganucleases, homing endonucleases, TALEN-based systems, or Zinc Finger Nucleases. The nanoparticle compositions may be used to deliver the nucleic acid (RNA and/or DNA) that encodes the sequences for these gene editing tools, and the actual gene products, proteins, or other molecules.

An embodiment comprises use of a nanoparticle composition herein for genome editing in a subject. An embodiment comprises a method of genome editing in a subject comprising administering a nanoparticle composition herein to the subject. The nucleic acid in these embodiments may be a sgRNA. The nucleic acid in these embodiments may be one for genome editing via meganucleases, homing endonucleases, TALEN-based systems, or Zinc Finger Nucleases.

In an embodiment, the nanoparticle composition may be used for gene targeting in a subject in vivo or ex vivo, e.g., by isolating cells from the subject, editing genes, and implanting the edited cells back into the subject. An embodiment comprises a method comprising administering a nanoparticle composition herein to isolated cells from a subject. The method may include gene targeting. The method may comprise implanting the edited cells back into the subject (or into another subject).

### EXAMPLES

The following non-limiting examples are provided to illustrate particular embodiments.

### Example 1. Nucleic Acid Carrier of Formula 1a: Polyester Dendrimer with Disulfide Core

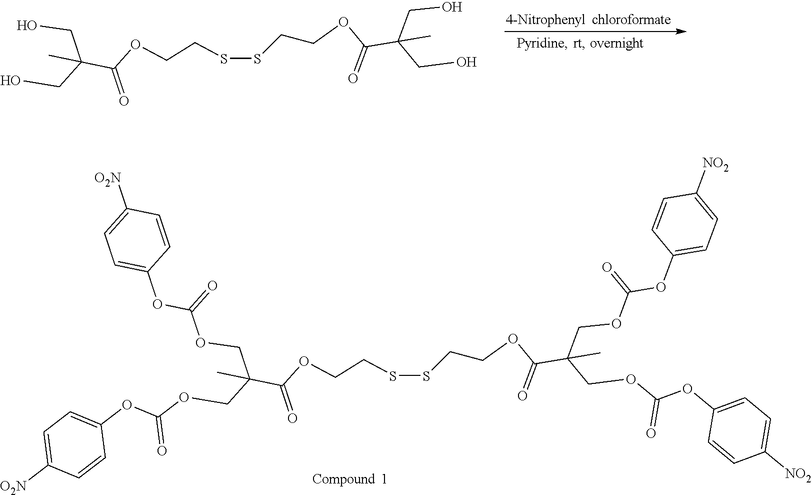

Compound 1: Bis-MPA hydroxyl dendrimer generation 1, disulfide core (250 mg, 0.647 mmol, MW 386.48) was dissolved in dry DCM (6 mL) and pyridine (0.94 ml, 11.6 mmol, 18 eq) was added followed by p-nitrophenyl chloroformate (2.2 g, 11 mmol, 17 eq, MW 201.6) dissolved in dry DCM (15 mL), the reaction mixture was stirred at 0° C. to 23° C. for 16 h, Next day TLC shows product formation. The reaction mixture was diluted with 1.33 M NaHSO4, extracted with EtOAc. The organic layer was washed with brine, dried over Na₂SO₄ and evaporated in Rotavap. The crude was then purified by flash chromatography with DCM/EtOAc. The compound eluted at 5% DCM/EtOAc, (R_{f}=0.8 in 10:1 DCM/EtOAc). Yield 500 mg (74%).

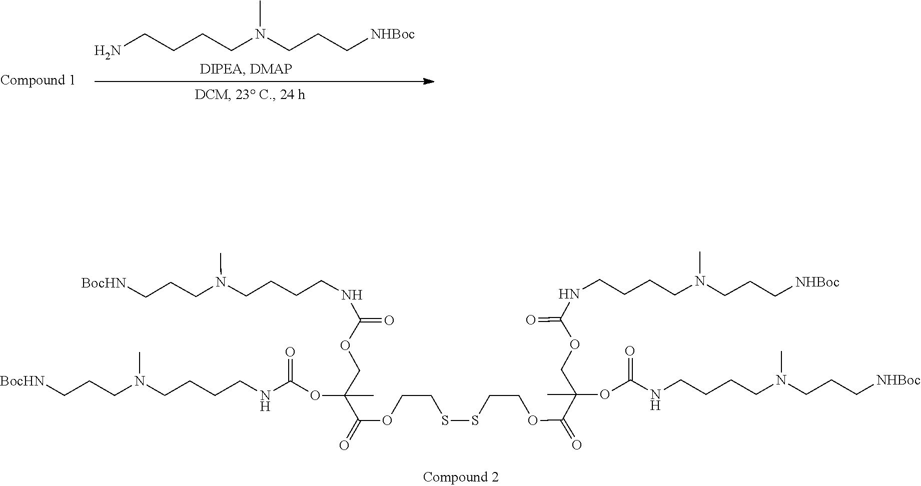 [SMILES: CN(CCCCN)CCCNC(=O)OC(C)(C)C ; CN(CCCCNC(=O)OCC(C)(OC(=O)NCCCCN(C)CCCNC(=O)OC(C)(C)C)C(=O)OCCSSCCOC(=O)C(C)(COC(=O)NCCCCN(C)CCCNC(=O)OC(C)(C)C)OC(=O)NCCCCN(C)CCCNC(=O)OC(C)(C)C)CCCNC(=O)OC(C)(C)C]

Compound 2: A solution of Compound 1 (167 mg, 0.16 mmol, MW 1046.13) dissolved in dry DCM (3 mL) was added to an excess of tert-butyl (3-((4-aminobutyl)(methyl)amino)-propyl)carbamate (207 mg, 0.8 mmol, 5 eq) dissolved in dry DCM (3 mL). A solution of DMAP (39 mg, 0.32 mmol) and DIPEA (0.11 ml, 0.64 mmol, MW 129.24, d 0.742) in dry DCM (1 mL) was added and the reaction mixture was stirred 24 h at 23° C. under an argon atmosphere. Next day the solvent was then removed in vacuo, purified via flash chromatography on silica column (24 g) with gradient elution from 100% CH₂Cl₂ (mobile phase a) to 75:22:3 CH₂Cl₂/MeOH/NH4OHaq (by volume, mobile phase b) over 40 minutes. The desired product eluted at 40% mobile phase b. Yield 195 mg (80%).

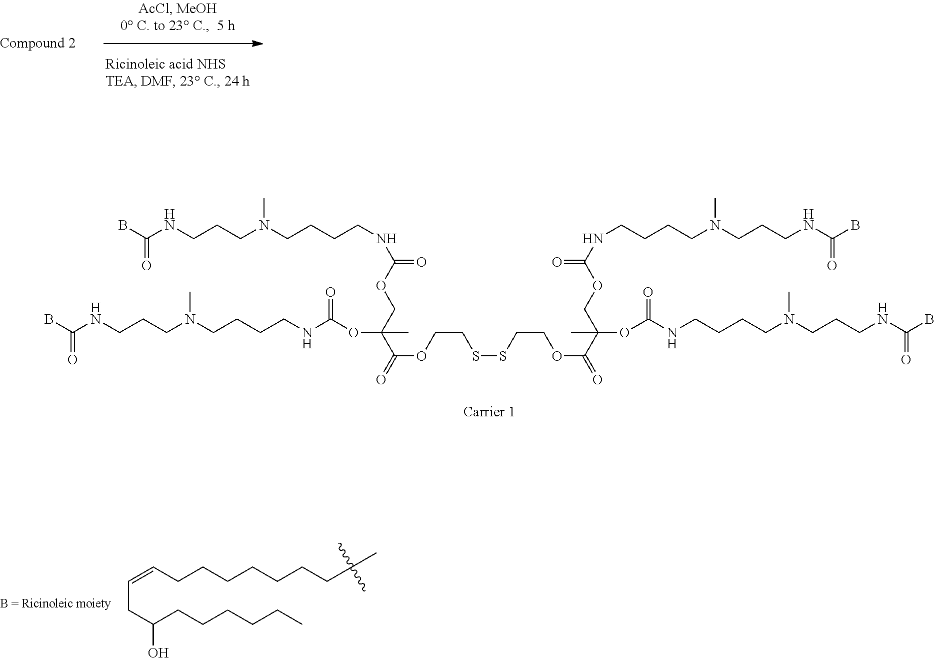

Carrier 1: 194 mg of Compound 2 (0.13 mmol) was treated with 20 eq of AcCl (0.18 ml, 2.54 mmol) after dissolving the compound in 3 ml MeOH, the reaction was stirred at 0° C. to 23° C. for 5 h, evaporated to dryness and the crude product was dissolved in 1 ml DMF, Et₃N (0.35 ml, 2.54 mmol) was added followed by Ricinoleic acid NHS ester³ (251 mg, 0.64 mmol, 6 eq) that was dissolved in 2.2 ml DMF. Next day the reaction mixture was concentrated and purified via flash chromatography on silica column (24 g) with gradient elution from 100% CH₂Cl₂ (mobile phase a) to 75:22:3 CH₂Cl₂/MeOH/NH₄OHaq (by volume, mobile phase b) over 40 minutes. The desired product eluted at 65% mobile phase b. (R_{f}=0.7 (2:1 mobile phase b/mobile phase a)) to yield the desired product as yellow oil (80 mg, 28%). MS (ESI) calcd for C₁₂₂H₂₃₀N₁₂O₂₀S₂ [M+2H]²⁺ m/z 1124.7, found 1125.5; [M+3H]³⁺ m/z 749.8, found 750.6.

### Example 2. Nucleic Acid Carrier of Formula 2b: Polyester Dendron with Disulfide in Focal Point

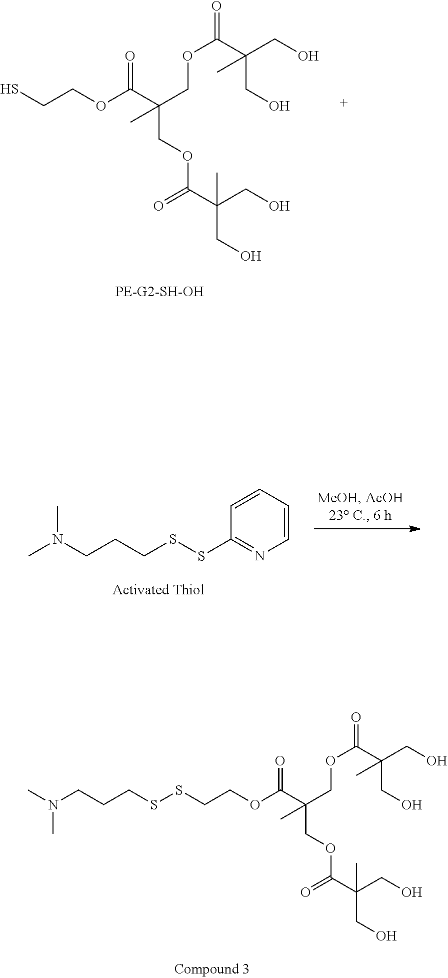 [SMILES: CC(CO)(CO)C(=O)OCC(C)(COC(=O)C(C)(CO)CO)C(=O)OCCS ; CN(C)CCCSSc1ccccn1 ; CN(C)CCCSSCCOC(=O)C(C)(COC(=O)C(C)(CO)CO)COC(=O)C(C)(CO)CO]

Compound 3: PE-G2-SH—OH (209 mg, 0.49 mmol, 1 equiv.) was dissolved in dry MeOH (3 mL) and dimethylaminopropyldisulfidylpyridine (112 mg, 0.49 mmol, 1 equiv.) dissolved in dry MeOH (1 mL) was added, the reaction mixture was stirred at 0° C. to 23° C. for 6 h. The reaction mixture was concentrated and purified via flash chromatography on silica gel column (24 g) with gradient elution from 100% CH₂Cl₂ (mobile phase a) to 75:22:3 CH₂Cl₂/MeOH/NH₄OHaq (by volume, mobile phase b) over 40 minutes. The desired product eluted at 36% mobile phase b. (R_{f}=0.6 in 1:1 mobile phase b/mobile phase a) to yield the desired product as yellow oil (85 mg, 31%).

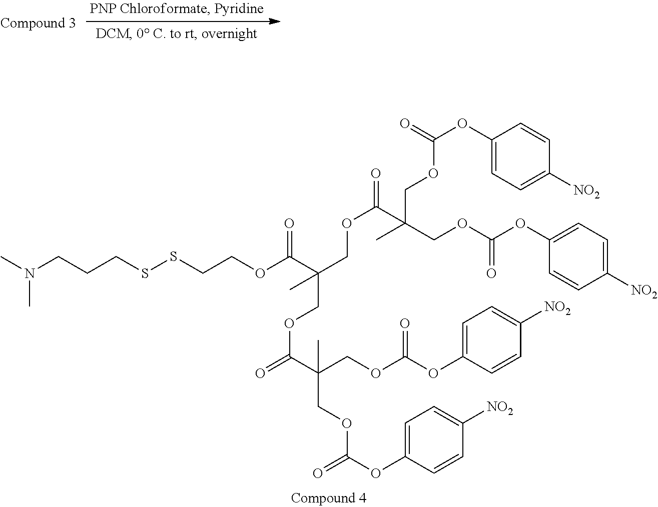 [SMILES: CN(C)CCCSSCCOC(=O)C(C)(COC(=O)C(C)(COC(=O)Oc1ccc([N+](=O)[O-])cc1)COC(=O)Oc1ccc([N+](=O)[O-])cc1)COC(=O)C(C)(COC(=O)Oc1ccc([N+](=O)[O-])cc1)COC(=O)Oc1ccc([N+](=O)[O-])cc1]

Compound 4: Compound 3 or PE-G2-S—S-PropylNMe2-OH (261 mg, 0.48 mmol) was dissolved in dry DCM (6 mL); Pyridine (0.73 ml, 9.06 mmol) was added followed by p-nitrophenyl chloroformate (1.7 g, 8.43 mmol) dissolved in dry DCM (15 mL), the reaction mixture was stirred at 0° C. to 23° C. for 16 h, Next day TLC shows product formation. The reaction mixture was diluted with 1.33 M NaHSO4, extracted with EtOAc. The organic layer was washed with brine, dried over Na₂SO₄ and evaporated in Rotavap. The crude was then purified by flash chromatography with DCM/MeOH. The compound eluted at 14% MeOH/DCM, (R_{f}=0.7 in 19:1 DCM/MeOH. Yield 319 mg (55%).

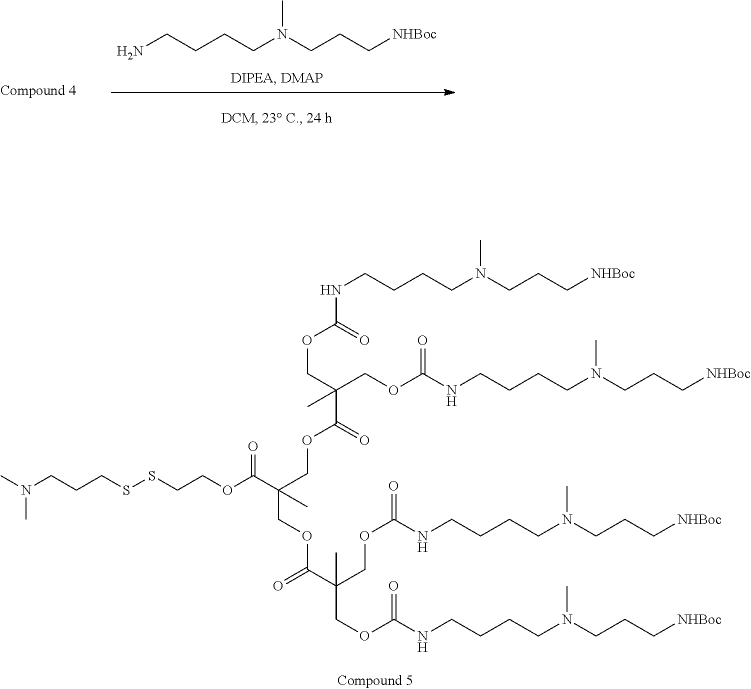 [SMILES: CN(CCCCN)CCCNC(=O)OC(C)(C)C ; CN(C)CCCSSCCOC(=O)C(C)(COC(=O)C(C)(COC(=O)NCCCCN(C)CCCNC(=O)OC(C)(C)C)COC(=O)NCCCCN(C)CCCNC(=O)OC(C)(C)C)COC(=O)C(C)(COC(=O)NCCCCN(C)CCCNC(=O)OC(C)(C)C)COC(=O)NCCCCN(C)CCCNC(=O)OC(C)(C)C]

Compound 5: A solution of Compound 4 (200 mg, 0.17 mmol) dissolved in dry DCM (3 mL) was added to an excess of tert-butyl (3-((4-aminobutyl)-(methyl)amino)propyl)carbamate (216 mg, 0.83 mmol) dissolved in dry DCM (3 mL). A solution of DMAP (41 mg, 0.33 mmol) and DIPEA (0.12 ml, 0.66 mmol, MW 129.24, d 0.742) in dry DCM (1 mL) was added and the reaction mixture was stirred 16 h at 23° C. under an argon atmosphere. Next day the solvent was then removed in vacuo, purified via flash chromatography on silica column (40 g) with gradient elution from 100% CH₂Cl₂ (mobile phase a) to 75:22:3 CH₂Cl₂/MeOH/NH4OHaq (by volume, mobile phase b) over 40 minutes. The desired product eluted at 50% mobile phase b. (silica gel TLC R_{f}=0.3 in 1:1 mobile phase a/mobile phase b). Yield 87 mg (31%).

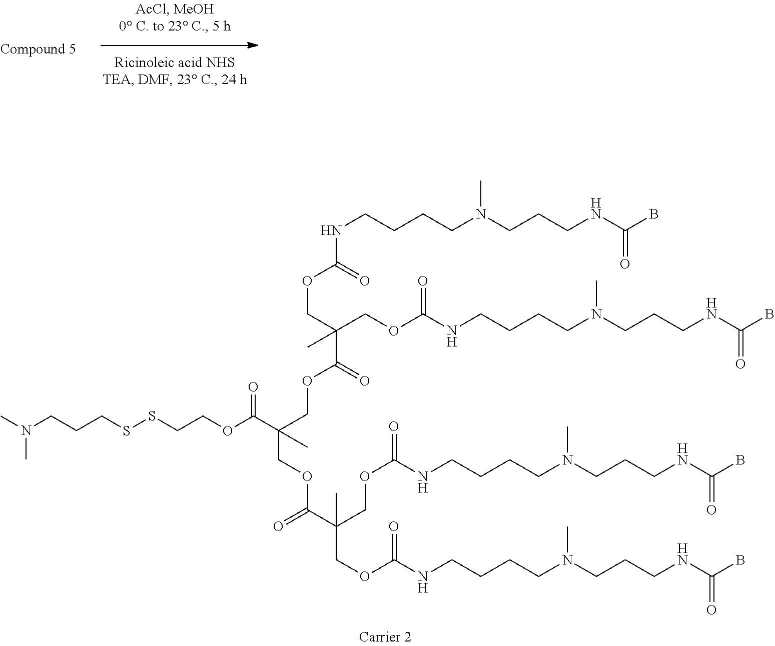

Carrier 2: Compound 5 (87 mg, 0.052 mmol) was treated with 20 eq of AcCl (0.074 ml, 1.03 mmol) after dissolving the compound in 3 ml MeOH, the reaction was stirred at 0° C. to 23° C. for 5 h, evaporated to dryness and the crude product was dissolved in 1.5 ml DMF, Et₃N (0.14 ml, 1.03 mmol) was added followed by Ricinoleic acid NHS ester (122 mg, 0.31 mmol, 6 eq) that was dissolved in 1.5 ml DMF. Next day the reaction mixture was concentrated and purified via flash chromatography on silica column (24 g) with gradient elution from 100% CH₂Cl₂ (mobile phase a) to 75:22:3 CH₂Cl₂/MeOH/NH₄OHaq (by volume, mobile phase b) over 40 minutes. The desired product eluted at 74% mobile phase b to yield the desired product as yellow oil (90 mg, 73%). MS (ESI) calcd for C₁₃₀H₂₄₅N₁₃O₂₂S₂[M+2H]²⁺ m/z 1203.3, found 1204.0; [M+3H]³⁺ m/z 802.1, found 803.0.

### Example 3: Nucleic Acid Carrier of Formula 3c: Polyester Dendron with Disulfide Based Hydrophobic Tails

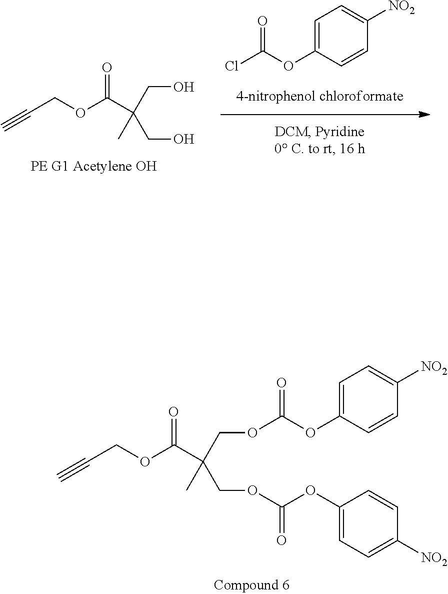 [SMILES: O=C(Cl)Oc1ccc([N+](=O)[O-])cc1 ; C#CCOC(=O)C(C)(CO)CO ; C#CCOC(=O)C(C)(COC(=O)Oc1ccc([N+](=O)[O-])cc1)COC(=O)Oc1ccc([N+](=O)[O-])cc1]

Compound 6: PE-G1-Acetylene-OH (641 mg, 3.73 mmol) was dissolved in dry DCM (6 mL) and (5.4 ml, 30 mmol) was added followed by p-nitrophenyl chloroformate (2 g, 10 mmol, 2.7 eq) dissolved in dry DCM (15 mL), the reaction mixture was stirred at 0° C. to 23° C. for 16 h, Next day TLC shows product formation. The reaction mixture was diluted with 1.33 M NaHSO4, extracted with EtOAc. The organic layer was washed with brine, dried over Na₂SO₄ and evaporated in Rotavap. The crude was then purified by flash chromatography with DCM/EtOAc. The compound eluted at DCM, (R_{f}=0.7 (19:1 DCM/EtOAc). Yield 765 mg (41%).

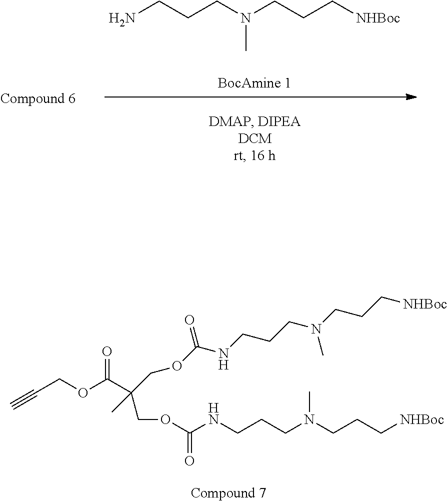 [SMILES: CN(CCCN)CCCNC(=O)OC(C)(C)C ; C#CCOC(=O)C(C)(COC(=O)NCCCN(C)CCCNC(=O)OC(C)(C)C)COC(=O)NCCCN(C)CCCNC(=O)OC(C)(C)C]

Compound 7: A solution of Compound 6 (350 mg, 0.70 mmol, MW 502.4) dissolved in dry DCM (3 mL) was added to an excess of BocAminel (513 mg, 2.09 mmol, MW 245.4) dissolved in dry DCM (6 mL). A solution of DMAP (170 mg, 1.39 mmol) and DIPEA (0.49 ml, 2.8 mmol) in dry DCM (1 mL) was added and the reaction mixture was stirred 16 h at 23° C. under an argon atmosphere. Next day the solvent was then removed in vacuo, purified via flash chromatography on silica column (40 g) with gradient elution from 100% CH₂Cl₂ (mobile phase a) to 75:22:3 CH₂Cl₂/MeOH/NH4OHaq (by volume, mobile phase b) over 40 minutes. The desired product eluted at 50% mobile phase b. (silica gel TLC R_{f}=0.55 in mobile phase a/mobile phase b). Yield 300 mg (60%).

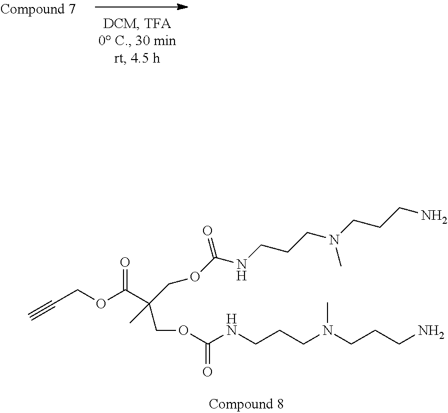 [SMILES: C#CCOC(=O)C(C)(COC(=O)NCCCN(C)CCCN)COC(=O)NCCCN(C)CCCN]

Compound 8: In a 20 mL clear glass vial with 160 mg of Compound 7 (0.16 mmol) was added 3.9 mL dry DCM. Reaction was cooled to 0° C. on ice bath. TFA (0.051 mL) was added dropwise. The ice bath was removed after 30 minutes, and the reaction stirred under Argon for 5 hours. The crude reaction was co-evaporated using DCM and MeOH to remove TFA. The crude Compound 8 was used directly in the coupling reaction of Compound 11.

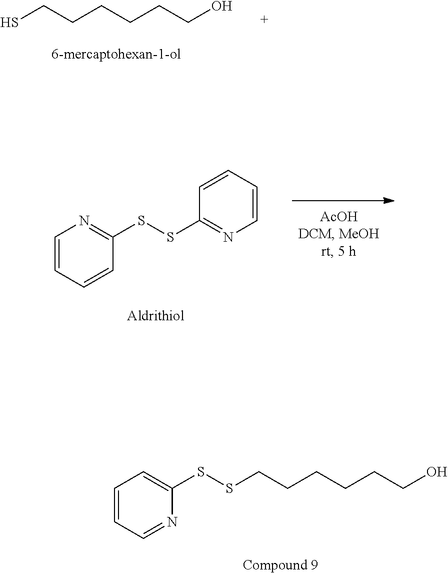 [SMILES: OCCCCCCS ; c1ccc(SSc2ccccn2)nc1 ; OCCCCCCSSc1ccccn1]

Compound 9: To a dry 50 mL RBF was added 1.64 g aldrithiol (7.44 mmol) and was then dissolved in 6 mL dry MeOH. Added 0.08 mL glacial acetic acid (0.93 mmol). Added 500 mg of 6-mercapto-1-hexanol (3.72 mmol) dissolved in 4 mL of dry DCM. The reaction was flushed with Argon and stirred at 23° C. for 5 hours. The solvent was then removed in vacuo. The crude was purified via flash chromatography on silica column (40 g) with gradient elution from 100% Hexanes (mobile phase a) to Ethyl Acetate (mobile phase b) over 1 hour. The desired product eluted at 30% mobile phase b. (silica get TLC R_{f}=0.2 in mobile phase a/mobile phase b). Yield 802 mg (88%). MS (ESI) calcd for C₁₁H₁₇NOS₂ [M+1]⁺ m/z 243, found 244.

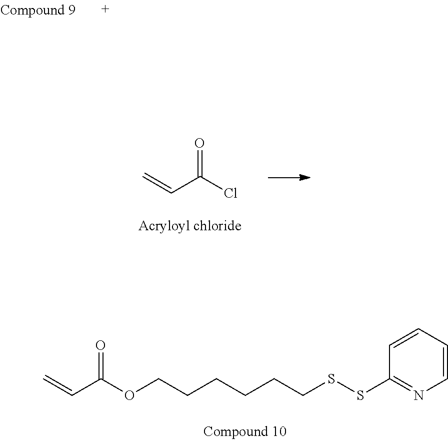

Compound 10: To dry 200 mL RBF with 600 mg of Compound 9 (2.47 mmol) was added 24 mL of dry DCM. Added 0.51 mL of triethylamine (3.70 mmol) was added. The reaction as cooled to 0° C. by ice bath and 0.24 mL of acryloyl chloride (3.03 mmol) was added dropwise. The ice bath was removed after 30 minutes and the reaction stirred overnight at 23° C. under an Argon atmosphere. The reaction was washed with water, followed by brine. The organic layer was collected and dried over Na₂SO₄ and filtered to a RBF. The solvent was removed in vacuo. The crude was purified via flash chromatography on silica column (24 g) with gradient elution from 100% Hexanes (mobile phase a) to Ethyl Acetate (mobile phase b) over 1 hour. The desired product eluted at 8% mobile phase b. (silica get TLC R_{f}=0.8 in mobile phase a/mobile phase b). Yield 360 mg (49%). MS (ESI) calcd for C₁₄H₁₉NO₂S2 [M]⁺ m/z 297.09, found 297.

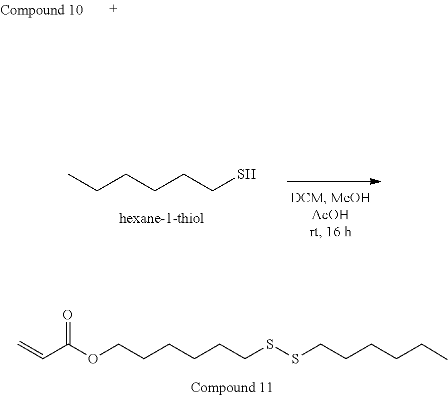

Compound 11: In a 20 mL clear glass vial with 205 mg of Compound 10 (0.69 mmol) was added 2 mL of dry DCM and 2 mL of dry MeOH. A catalytic amount of glacial acetic acid was added. Hexanethiol (0.15 mL, 1.03 mmol) was added dropwise. The reaction was flushed with Argon and stirred overnight at 23° C. The next day the solvent was then removed in vacuo. The crude was purified via flash chromatography on silica column (24 g) with gradient elution from 100% Hexanes (mobile phase a) to Ethyl Acetate (mobile phase b) over 26 minutes. The desired product eluted at 6% mobile phase b. (silica gel TLC R_{f}=0.65 in mobile phase a/mobile phase b). Yield 88 mg (42%).

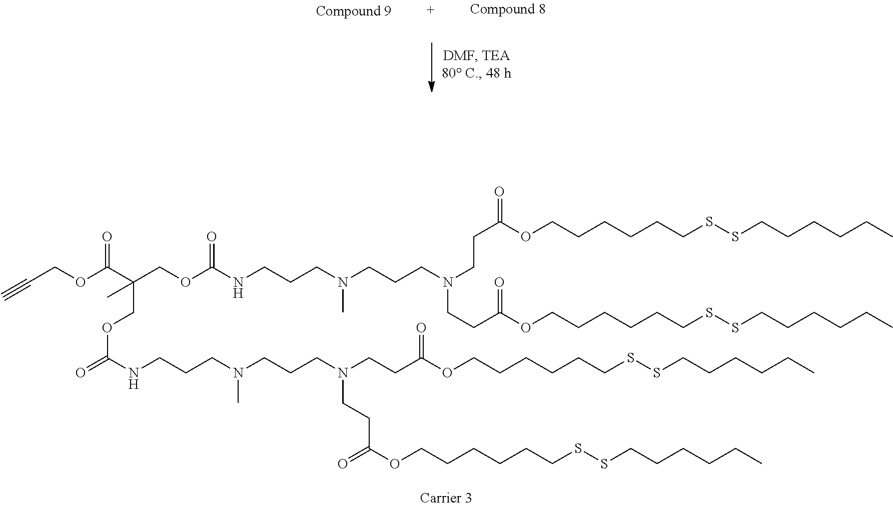 [SMILES: C#CCOC(=O)C(C)(COC(=O)NCCCN(C)CCCN(CCC(=O)OCCCCCCSSCCCCCC)CCC(=O)OCCCCCCSSCCCCCC)COC(=O)NCCCN(C)CCCN(CCC(=O)OCCCCCCSSCCCCCC)CCC(=O)OCCCCCCSSCCCCCC]

Carrier 3: To 20 mL clear glass vial with 58 mg of Compound 8 (0.11 mmol) was added 0.5 mL dry DMF. Added 0.47 mL of triethylamine (0.34 mmol) to neutralize TFA. The reaction was allowed to stir for 10 minutes under Argon at 23° C. Added 152 mg of Compound 9 (0.5 mmol) dissolved in 0.5 mL of dry DMF. Flushed the reaction with argon and stirred at 80° C. for 48 hours. After 48 hours the reaction crude was purified via flash chromatography on silica column (24 g) with gradient elution from 100% CH₂Cl₂ (mobile phase a) to 75:22:3 CH₂Cl₂/MeOH/NH₄OHaq (by volume, mobile phase b) over 76 minutes. The desired product eluted at 12% mobile phase b. (silica gel TLC R_{f}=0.50 in mobile phase a/mobile phase b). Yield 28 mg (14%). MS (ESI) calcd for C84H158N6014S8 [M/3]+ m/z 577, found 578.3, [M/2]+ m/z 865.5, found 867.1.

### Example 4. Nucleic Acid Carrier of Formula 4a: Poly-Disulfide Dendron with Acetylene Core

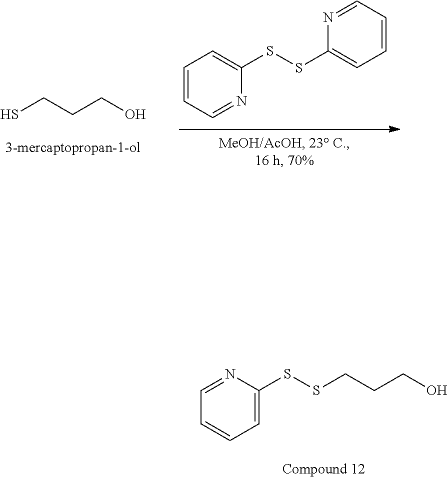 [SMILES: OCCCS ; c1ccc(SSc2ccccn2)nc1 ; OCCCSSc1ccccn1]

Compound 12: 1.07 g of Aldrithiol (4.87 mmol, 1.5 eq) was dissolved in 6 mL of anhydrous methanol and 300 mg (3.25 mmol) of 3-mercaptopropanol was added dropwise to the reaction, followed by 100 μL of glacial acetic acid. The reaction was stirred at 23° C. under an argon atmosphere for 16 hours. Then, the solvent was removed in vacuo, and the crude product was purified via flash chromatography on silica column (12 g) with gradient elution from 100% Hexanes (mobile phase a) to 100% Ethyl Acetate (by volume, mobile phase b) over 35 minutes. The desired product eluted at 30% mobile phase b. (silica gel TLC R_{f}=0.21 in 3:2 Hexane:EtOAc). Yield 464 mg (70%). MS (ESI) calcd for C₈H₁₁NOS₂ [M+H]⁺ m/z 202, found 202.

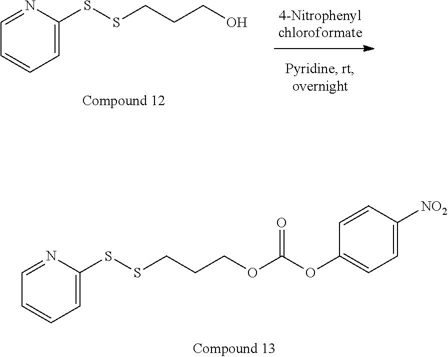 [SMILES: OCCCSSc1ccccn1 ; O=C(OCCCSSc1ccccn1)Oc1ccc([N+](=O)[O-])cc1]

Compound 13: Compound 12 (464 mg, 2.3 mmol) was dissolved in anhydrous DCM (4 mL) and 0.34 ml of pyridine (30 mmol, 2 eq) was added. The reaction was cooled to 0° C. and p-nitrophenyl chloroformate (0.695 g, 3.44 mmol, 1.5 eq) dissolved in dry DCM (4 mL) was added dropwise to the reaction. The reaction mixture was stirred at 0° C. to 23° C. for 16 h, Next day the reaction mixture was diluted with 1.33 M NaHSO4, extracted with DCM. The organic layer was washed with brine, dried over Na₂SO₄ and evaporated in Rotavap. The crude was then purified by flash chromatography (24 g) with DCM/EtOAc. The compound eluted at 78% DCM, (R_{f}=0.21 in DCM). Yield 410 mg (49%). MS (ESI) calcd for C₁₅H₁₄N₂O₅S₂[M+H]⁺ m/z 367, found 368.

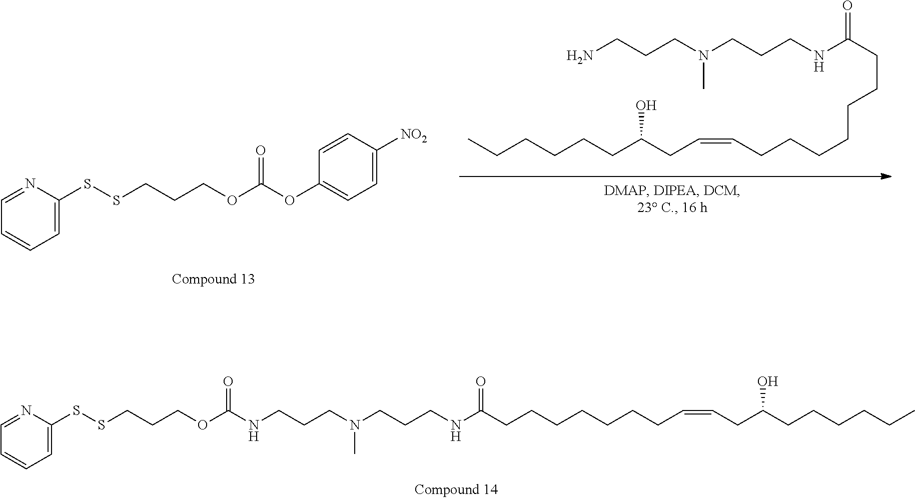 [SMILES: O=C(OCCCSSc1ccccn1)Oc1ccc([N+](=O)[O-])cc1 ; CCCCCC[C@H](O)C/C=C\CCCCCCCC(=O)NCCCN(C)CCCN ; CCCCCC[C@@H](O)C/C=C\CCCCCCCC(=O)NCCCN(C)CCCNC(=O)OCCCSSc1ccccn1]

Compound 14: A solution of Compound 13 (100 mg, 0.272 mmol, MW 367) dissolved in dry DCM (2 mL) was added to an excess of (S,Z)-N-(3-((3-aminopropyl)-(methyl)amino)propyl)-12-hydroxyoctadec-9-enamide (131 mg, 0.3 mmol, 1.1 eq, MW 439.7), (synthesized as reported in WO/2025/096681) dissolved in dry DCM (6 mL). A solution of DMAP (49 mg, 0.408 mmol, 1.5 eq) and DIPEA (0.071 ml, 0.408 mmol, 1.5 mmol) in dry DCM (2 mL) was added and the reaction mixture was stirred 16 h at 23° C. under an argon atmosphere. Next day the solvent was removed in vacuo, purified via flash chromatography on silica column (12 g) with gradient elution from 100% CH₂Cl₂ (mobile phase a) to 75:22:3 CH₂Cl₂/MeOH/NH4OHaq (by volume, mobile phase b) over 40 minutes. The desired product eluted at 16% mobile phase b. (silica gel TLC R_{f}=0.54 in 1:1 mobile phase a/mobile phase b). Yield 110 mg (62%).

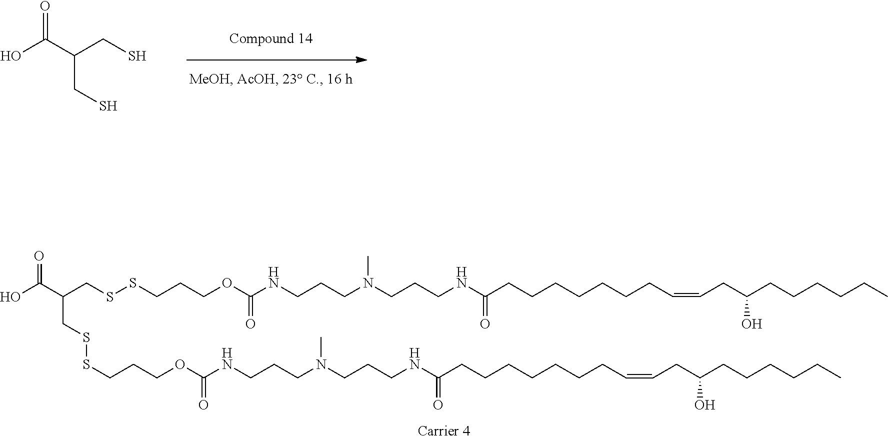 [SMILES: O=C(O)C(CS)CS ; CCCCCC[C@H](O)C/C=C\CCCCCCCC(=O)NCCCN(C)CCCNC(=O)OCCCSSCC(CSSCCCOC(=O)NCCCN(C)CCCNC(=O)CCCCCCC/C=C\C[C@@H](O)CCCCCC)C(=O)O]

Carrier 4: 110 mg (0.169 mmol, 2.2 eq) of Compound 14 was dissolved in 5 mL anhydrous methanol, and 12 mg of 3-mercapto-2-(mercaptomethyl)propanoic acid (0.0766 mmol, 1 eq, Exact Mass: 152.00) dissolved in 1 mL MeOH was added to it, followed by 100 μL of glacial acetic acid. Reaction was stirred at 23° C. under argon atmosphere for 16 hours. Next day the solvent was removed in vacuo, purified via flash chromatography on silica column (24 g) with gradient elution from 100% CH₂Cl₂ (mobile phase a) to 75:22:3 CH₂Cl₂/MeOH/NH4OHaq (by volume, mobile phase b) over 62 minutes. The desired product eluted at 70% mobile phase b. (silica gel TLC R_{f}=0.133 in 1:1 mobile phase a/mobile phase b). Yield 22 mg (11%). MS (ESI) calcd for C₆₂H₁₁₈N₆O₁₀S₄ [M+H]⁺ m/z 1235.8, found 1235.6. ¹H NMR (400 MHz, CHLOROFORM-D) δ 7.20 (s, 1H), 6.17 (s, 1H), 5.55-5.46 (m, 2H), 5.43-5.32 (m, 2H), 4.13 (m, 4H), 3.64-3.53 (m, 2H), 3.34-3.29 (m, 4H), 3.27 (m, 3H), 3.18-3.01 (m, 12H), 2.98-2.92 (m, 2H), 2.85-2.79 (m, 4H), 2.76 (s, 6H), 2.19 (m, 8H), 2.00 (m, 16H), 1.58 (m, 4H), 1.48-1.39 (m, 6H), 1.27 (d, m, 34H), 0.94-0.81 (m, 6H).

### Nanoparticle Formulation Containing Nucleic Acid Carriers of Formula 1a and 2a:

Nanoparticles containing Carrier 1 or Carrier 2:cholesterol:DOPE:DMG-PEG2k at molar ratios of 1:2.88:0.6:0.75 were formulated using NanoAssemblr Benchtop (Precision NanoSystems Inc, Vancouver, BC, Canada)). RNA was diluted with DNase/RNase-Free, endotoxin-free distilled water and sterile acetate buffer to a final desired pH. Total flow rate was maintained at 8 mL per min at a 3:1 ratio of aqueous to organic phase for formulating on the Benchtop. Using glassware depyrogenated by heating at 250° C. for 24 hours, nanoparticles were dialyzed against sterile, endotoxin-free PBS using 20,000 molecular weight cutoff dialysis. Dialyzed nanoparticles were sterile filtered using 0.2-micron poly(ether sulfone) filters.

### Hydrodynamic Size, Polydispersity Index(PDI) and Encapsulation Efficiency Data

The “Z average” of the nanoparticle composition containing compound Carrier 1 or Carrier 2 and Luciferase Ψ mRNA as function of size was determined by dynamic light scattering (DLS). Encapsulation efficiency was measured by RiboGreen® assay. The nanoparticles showed monomodal size distribution, a PDI below 0.2, and very high encapsulation efficiency (EE).

| | | | Encapsulation |
|---|---|---|---|
| | Z-Ave | Polydispersity | efficiency |
| Sample Name | (d · nm) | Index (PdI) | (EE %) |
| Carrier 1.Luc Ψ mRNA | 101.9 | 0.067 | 97 |
| Carrier 2.Luc Ψ mRNA | 109.4 | 0.074 | 98 |

### Agarose Gel Electrophoresis

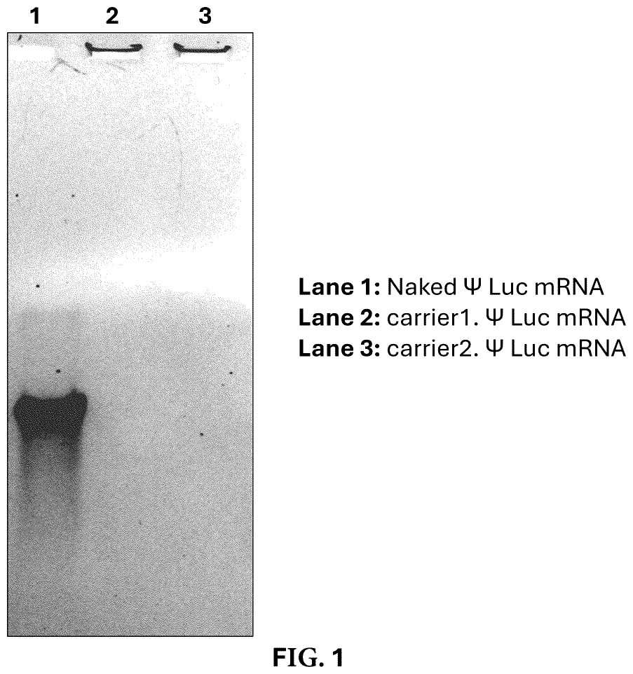

The concentration of RNA was determined by NanoDrop measurement (Thermo Scientific). Agarose gel electrophoresis was performed to evaluate the binding of nucleic acid carrier with RNA. FIG. 1 is a photograph of the agarose gel demonstrating the binding of the carriers with RNA. Referring to FIG. 1, lane 1 contained the unformulated Luc ΨmRNA, lane 2 contained the product of formulation of the carrier 1 and Luc ΨmRNA, lane 3 contained the product of formulation of the carrier 2 and Luc ΨmRNA. Before loading, the samples were incubated with formaldehyde loading dye containing ethidium bromide, denatured for 10 min at 65° C. and cooled to room temperature. The gel was run at 90 V and gel images were taken on a Syngene G Box imaging system (Syngene, USA). Referring to FIG. 1, the lower band corresponds to the small size free RNA (lane 1) and the top bands associated with the loading wells of the gel represent the nanoparticles formed by binding of the RNA to the dendrimer carriers.

### In Vitro Luciferase Expression Results

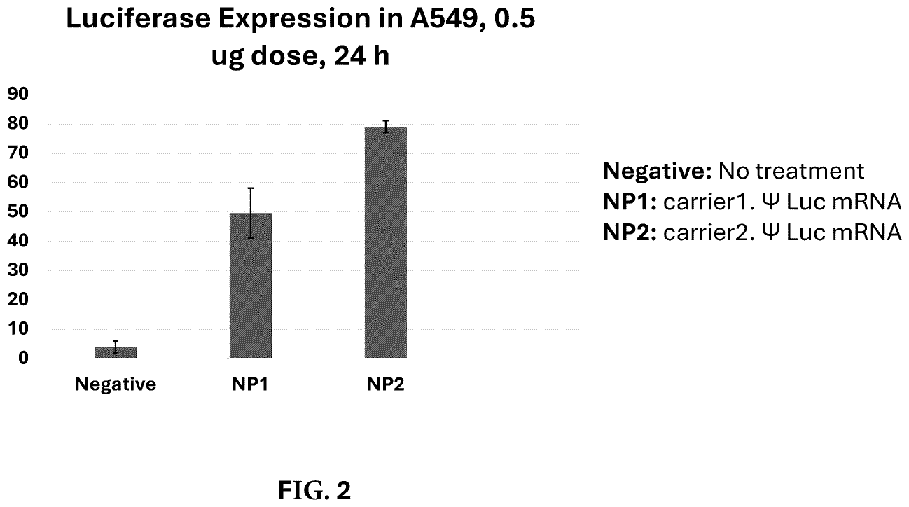

Now referring to FIG. 2, A549 (ATCC, CCL-185) cells were grown and maintained according to the manufacturer's recommended protocols. To test newly formulated nanoparticles for their ability to deliver mRNA into mammalian cells and drive protein expression, monolayers of A549 cells grown in 12-well plate were transfected 10 μL (approximately 1 μg worth of RNA) of each formulation product diluted into a final volume of 500 μL with a 1:1 OptiMEM:PBS mix. After the treatment, A549 cells were incubated at 37° C. and 5% CO2. After 12, hours, cell culture medium was collected, cells were lysed and luciferase activity was measured using luciferase one-step glow assay kit (ThermoFisher, #88263). FIG. 2 shows luciferase expression in cell culture from luciferase mRNA in disulfide-based polyester dendrimer nanoparticles delivered into mammalian cells and measured by quantification of intracellular luciferase activity using the luminescence assay. Referring to FIG. 2, it was observed that all the RNA nanoparticles were able to be taken up by A549 cells leading to gene expression.

### Biodistribution Study

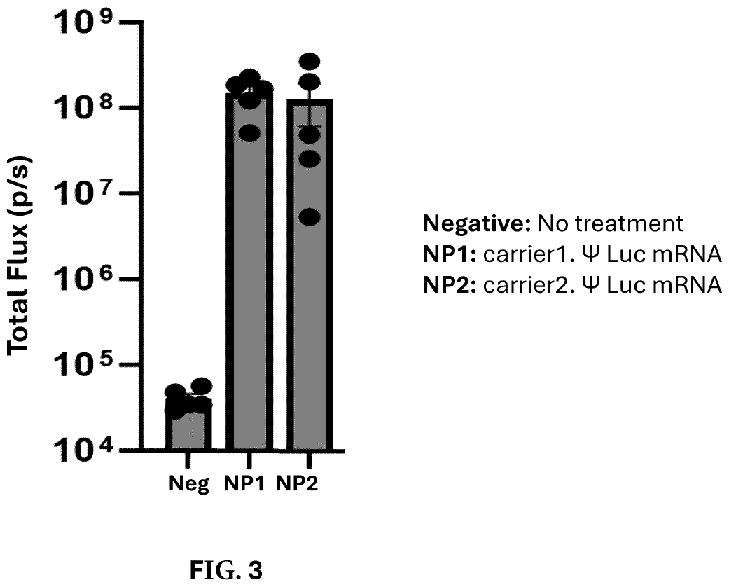

The nanoparticle composition based on Carrier 1 or Carrier 2 and Luciferase Ψ mRNA were given to mice by i.v. injection at the dose of 10 μg per animal. At 6-hour post injection, the D-luciferin reagent was administered to the mice by subcutaneous injection. The mice were imaged using an IVIS live-animal imaging system (Perkin Elmer) ˜10 min after D-luciferin injection and total luciferase expression was measured (FIG. 3). Based on the biodistribution data of Luciferase Ψ mRNA nanoparticles formulated with carrier 1 and carrier 2, 90% of total flux was found in lungs for carrier 1 and 87% of total flux was found in lungs for carrier 2.

The references cited throughout this application, are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

1. A compound of formula I: 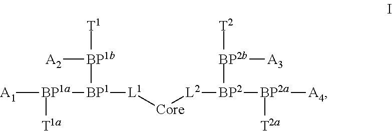
in which
Core is disulfide or C₁-C₆ alkylene unsubstituted or substituted with one or more of carboxyl, carboxylate, carbamate, and carbamide;
each of L¹ and L², independently, is C₁-C₃ alkylene;
each of BP¹ and BP², independently, is a bond, disulfide, O, S, NH, C₁-C₆ alkyl-N, N, or 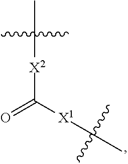
X¹ being a bond, NH, C₁-C₆ alkyl-N, O, S, or SS, X² being NH, C₁-C₆ alkyl-N, N, O, S, SS, SS—(CH₂)₁₋₂₀O, 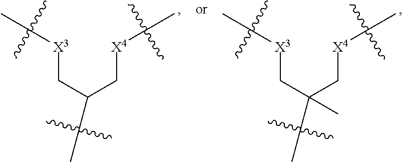
and each of X³ and X⁴, independently, being NH, C₁-C₆ alkyl-N, O, S, SS, or SS—(CH₂)₁₋₂₀O;
each of BP^{1a} and BP^{2a} is absent, a bond, disulfide, O, S, NH, C₁-C₆ alkyl-N, N, or 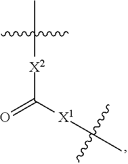
X¹ being a bond, NH, C₁-C₆ alkyl-N, O, S, or SS, X² being NH, C₁-C₆ alkyl-N, N, O, S, SS, SS—(CH₂)₁₋₂₀O, 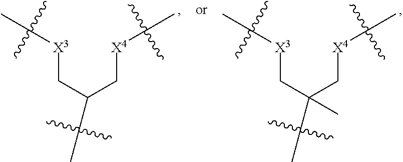
and each of X³ and X⁴, independently, being NH, C₁-C₆ alkyl-N, O, S, SS, or SS—(CH₂)₁₋₂₀O;
each of BP^{1b} and BP^{2b} is a bond, disulfide, O, S, NH, C₁-C₆ alkyl-N, N, or 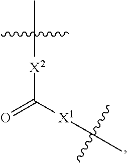
X¹ being a bond, NH, C₁-C₆ alkyl-N, O, S, or SS, X² being NH, C₁-C₆ alkyl-N, N, O, SS, SS—(CH₂)₁₋₂₀O, 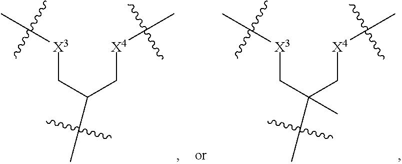
and each of X³ and X⁴, independently, being NH, C₁-C₆ alkyl-N, O, S, SS, or SS—(CH₂)₁₋₂₀O;
each of T^{1a} and T^{2a}, independently, is absent, 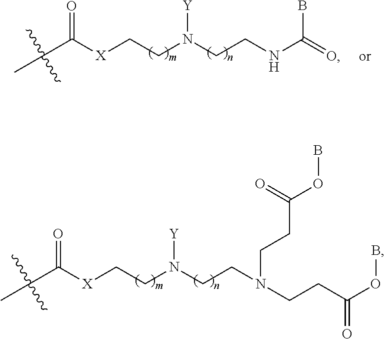
wherein B is a hydrophobic unit; X is NH, O, or S; Y is H, CH₃, C₂H₅, C₃H₇, CH₂CH₂OH, CH₂CH₂CH₂OH, CH₂CH₂NMe₂, CH₂CH₂CH₂NMe₂, CH₂CH₂O(CO)NHMe, or CH₂CH₂CH₂O(CO)NHMe; and each of m and n, independently, is 0 to 20,
each of T¹ and T², independently, is 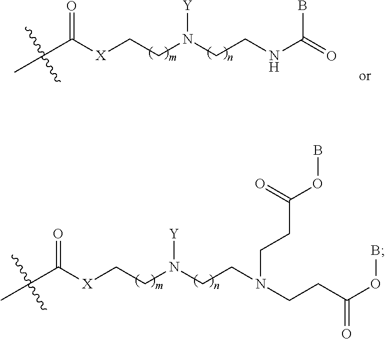
and
each of A, A², A³, and A⁴, independently, is absent, an amine,

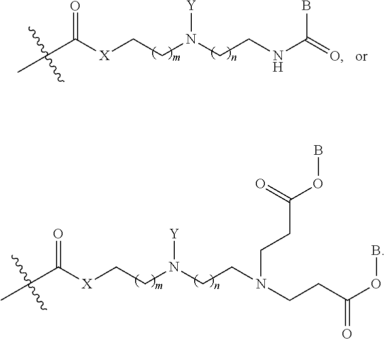

2. The compound of claim 1, wherein Core is S—S, 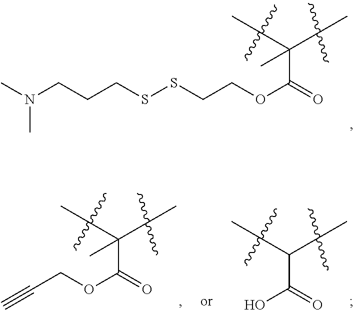
BP¹ is a bond, or 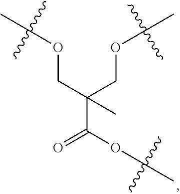 [SMILES: *OCC(C)(CO*)C(=O)O*]
BP² is 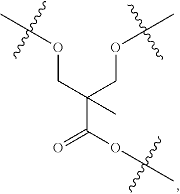 [SMILES: *OCC(C)(CO*)C(=O)O*]
BP^{1b} is absent, a bond, or 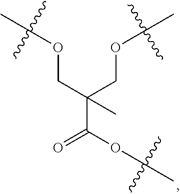 [SMILES: *OCC(C)(CO*)C(=O)O*]
each of BP^{1a}, BP^{2a} and BP^{2b} is a bond, or 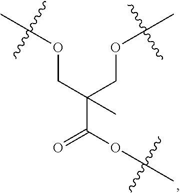 [SMILES: *OCC(C)(CO*)C(=O)O*]
provided that (i) when each of BP^{1a} and BP^{1a} is a bond, A¹ and A² are absent and T^{1a} is 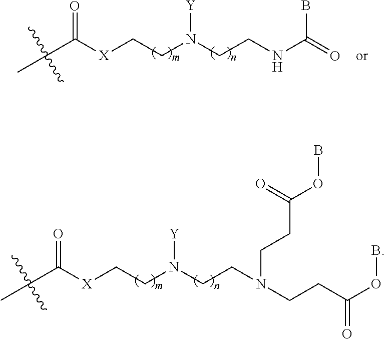
and (ii) when each of BP^{2a} and BP^{2b} is a bond, A³ and A⁴ are absent and T^{2a} is

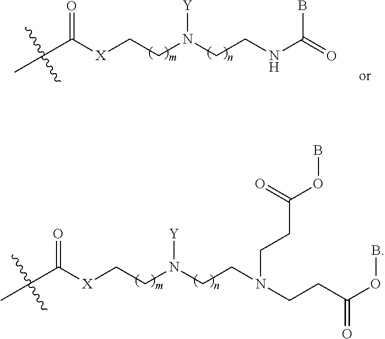

3. The compound of claim 1, wherein each of L¹ and L² is methylene or ethylene.

4. The compound of claim 1, wherein each of BP^{1a}, BP^{1b}, BP^{2a}, and BP^{2b} is a bond; each of T¹, T^{1a}, T², T^{2a}, independently, is 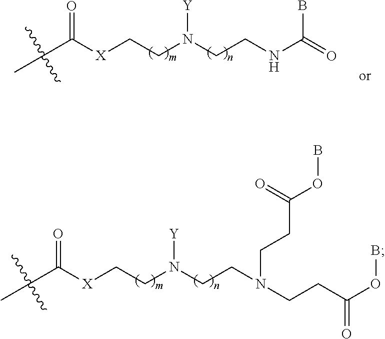
each of A¹, A², A³, and A⁴ is absent; and B is a disulfanyl or fatty acid tail.

5. The compound of claim 1, wherein T¹, T^{1a}, A², and BP^{1b} is absent, each of BP¹ and BP¹a is a bond, each of BP^{2a} and BP^{2b} is a bond or 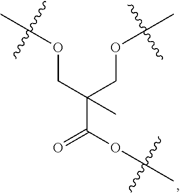 [SMILES: *OCC(C)(CO*)C(=O)O*]
each of T² and T^{2a}, independently, is 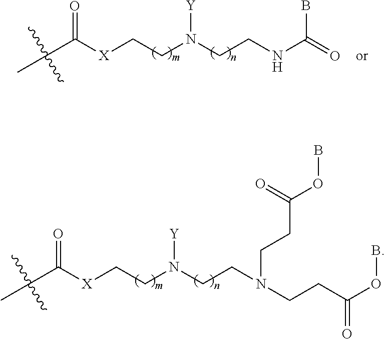
and each of A³ and A⁴ are absent,

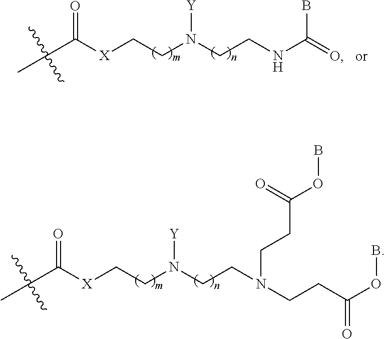

6. The compound of claim 1, wherein X is NH.

7. The compound of claim 1, wherein Y is CH₃.

8. The compound of claim 1, wherein B is a ricinoleyl moiety.

9. The compound of claim 1, wherein m is 3 and n is 2.

10. The compound of claim 1, wherein the compound is one of Carriers 1-4

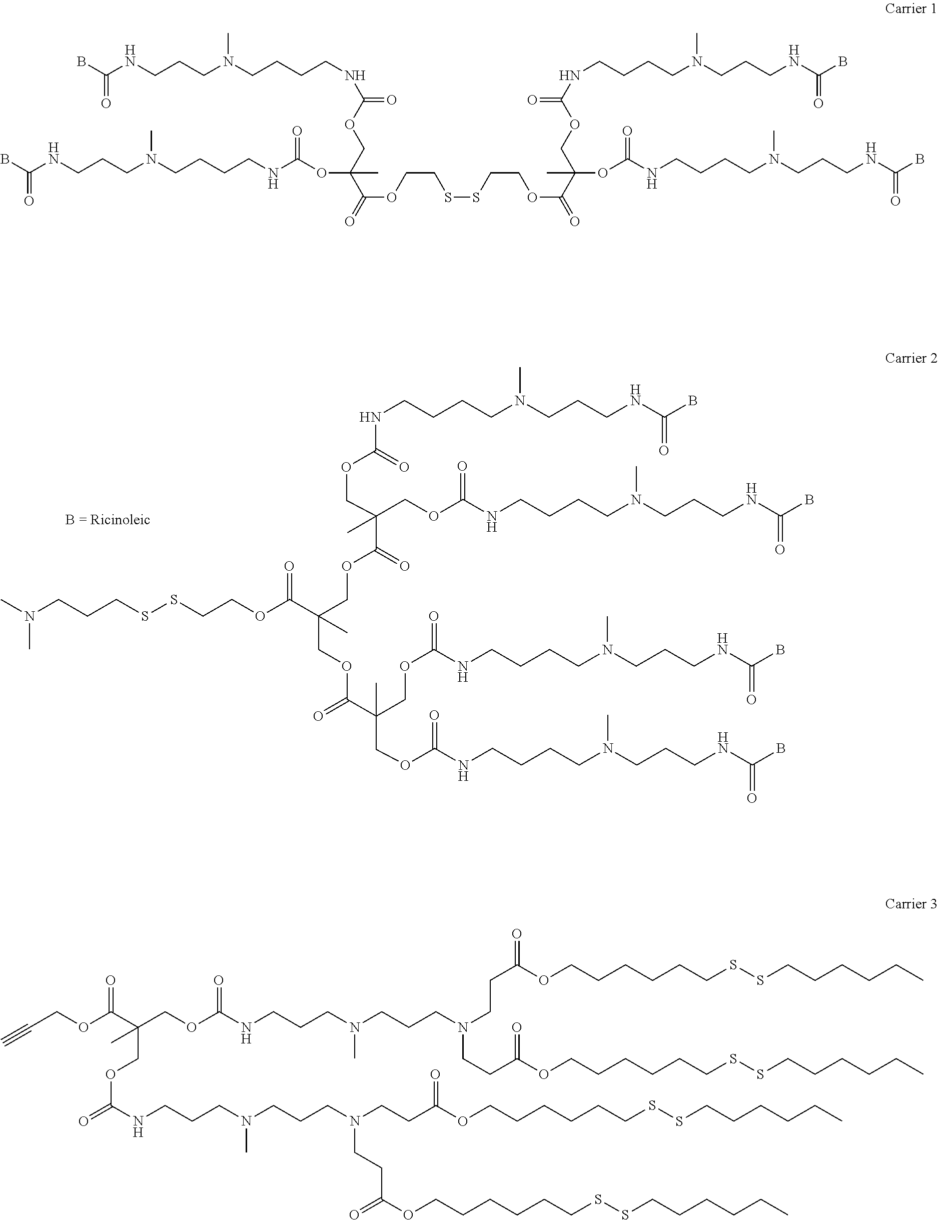

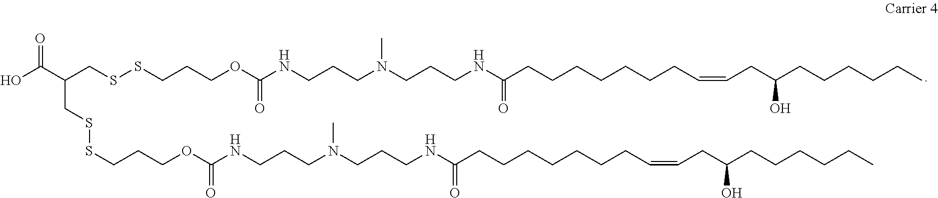 [SMILES: CCCCCC[C@@H](O)C/C=C\CCCCCCCC(=O)NCCCN(C)CCCNC(=O)OCCCSSCC(CSSCCCOC(=O)NCCCN(C)CCCNC(=O)CCCCCCC/C=C\C[C@H](O)CCCCCC)C(=O)O]

11. A nanoparticle composition comprising a compound of claim 1 and a nucleic acid agent, wherein the nucleic acid agent is therapeutic or immunogenic fully or partially encapsulated in the nanoparticle.

12. The nanoparticle composition of claim 11, wherein the therapeutic or immunogenic nucleic acid agent is selected from the group consisting of: a polynucleotide, oligonucleotide, DNA, cDNA, RNA, repRNA, siRNA, miRNA, sgRNA, and mRNA.

13. The nanoparticle composition of claim 11, wherein the therapeutic or immunogenic nucleic acid agent encodes one or more proteins or antigens associated with a condition selected from the group consisting of infectious disease, pathogen, cancer, autoimmunity disease and allergenic disease.

14. The nanoparticle composition of claim 11, wherein the therapeutic or immunogenic nucleic acid agent comprises an RNA or DNA capable of silencing, inhibiting or modifying the activity of a gene.

15. The nanoparticle composition of claim 11, further comprising a PEG-lipid.

16. The nanoparticle composition of claim 11, further comprising a PEG-lipid, preferable the PEG-lipid is 1,2-dimyristoyl-sn-glycero-3-phosphoethanol-amine-N-[methoxy (poly-ethylene glycol)-2000], or 1,2-dimyristoyl-rac-glycero-3-methoxypolyethylene glycol-2000.

17. The nanoparticle composition of claim 16, wherein the nanoparticle composition comprises the PEG-lipid in a range from 1 mol % to 10 mol % of the PEG-lipid per nanoparticle composition.

18. The nanoparticle composition of claim 11, further comprising at least one of a phospholipid, cholesterol, and a cholesterol derivative.

19. The nanoparticle composition of claim 18, wherein the phospholipid is 1,2-dioleoyl-sn-glycero-3-phosphoethanolamine (DOPE) or distearoylphosphatidylcholine (DSPC).

20. The nanoparticle composition of claim 18, wherein the nanoparticle composition comprises the phospholipid in a range from 10 mol % to 15 mol % of the nanoparticle composition where mol % of phospholipid=(mol of phospholipid/[mol of phospholipid+mol of cholesterol or the cholesterol derivative+mol of PEG lipid+mol of nucleic acid carrier+mol of nucleic acid])×100.

21. The nanoparticle composition of claim 18, wherein the nanoparticle composition comprises cholesterol or the cholesterol derivative in a range from 50 mol % to 75 mol % of the nanoparticle composition where mol % of cholesterol or the cholesterol derivative=(mol of cholesterol or the cholesterol derivative/[mol of cholesterol or the cholesterol derivative+mol of phospholipid+mol of PEG lipid+mol of nucleic acid carrier+mol of nucleic acid])×100.

22. A method for treating or preventing a disease or condition in a subject comprising the step of administering a therapeutically effective amount of a nanoparticle composition of claim 11 to the subject.

23. The method of claim 22, wherein the therapeutically effective amount of the nanoparticle composition comprises the therapeutic or immunogenic nucleic acid agent in a range from 0.01 mg nucleic acid to 10 mg nucleic acid per kg body weight of the subject.

24. The method of claim 22, wherein the subject is a mammal selected from the group consisting of a rodent, a canine, a primate, an equine, a high-value agricultural animal, and a human.
